(19) United States
(12) Patent Application Publication
(10) Pub. No.: US 2017/0265745 A1
(43) Pub. Date: Sep. 21, 2017

(54) INTEGRATED OPTICAL COHERENCE TOMOGRAPHY (OCT) SCANNING AND/OR THERAPEUTIC ACCESS TOOLS AND METHODS

(71) Applicant: COLLAGE MEDICAL IMAGING LTD., Beer-Sheva (IL)

(72) Inventors: Gavriel J. IDDAN, Haifa (IL); Roni ZVULONI, Haifa (IL)

(21) Appl. No.: 15/500,061

(22) PCT Filed: Jul. 29, 2015
(86) PCT No.: PCT/IL15/50782

§ 371 Date: Jan. 29, 2017

Related U.S. Application Data
(60) Provisional application No. 62/030,131, filed on Jul. 29, 2014.
(60) Provisional application No. 62/164,610, filed on May 21, 2015.

(51) Int. Cl. A61B 5/00; A61B 10/02

(52) U.S. Cl. CPC A61B 5/0066; A61B 10/0233; A61B 5/6848; A61B 5/0084

(57) ABSTRACT

The present application in some embodiments relates to methods for directing a procedure in a region of interest (ROI) using an access tube for introducing an optical coherence tomography (OCT) scanning head into the ROI and/or scanning the ROI with the scanning head. Optionally the scanning head is retracting from ROI via the access tube while the distal end of the access tube remains in the ROI and a second tool is introduced into ROI via the access tube. An access tube may include a standard cannula and/or some or all of a distal opening, a proximal opening and/or a window. All or part of an OCT engine and/or a control system (for example including a location indicator for 3D collage image composition) may be attached to the tool. In some embodiments an OCT tool, control system and/or OCT engine may be fully retractable from the access tube.

### RELATED APPLICATION/S

This application claims the benefit of priority under 35 USC §119(e) of U.S. Provisional Patent Application No. 62/030,131 filed 29 Jul. 2014, the contents of which are incorporated herein by reference in their entirety.

This application claims the benefit of priority under 35 USC §119(e) of U.S. Provisional Patent Application No. 62/164,610 filed 21 May 2015, the contents of which are incorporated herein by reference in their entirety.

### FIELD AND BACKGROUND OF THE INVENTION

The present invention, in some embodiments thereof, relates to a biopsy access tube, tools and/or methods, and, more particularly, but not exclusively, to tools and/or methods for sharing a access tube for multi-dimensional Optical Coherence Tomography scanning and other procedures.

U.S. Patent Publication no. 2015/0173619 discloses, “Systems and methods for scanning an organ or other extended volumes of body tissue using one or more Optical Coherence Tomography (OCT) probes are presented. Some embodiments

provide equipment for managing a plurality of OCT penetrations into a tissue or organ, and provide some or all of the following: detection and/or control of OCT probe positions and orientations (and optionally, that of other imaging modalities) detecting changes in body tissue positions, registering and mapping OCT scan results and optionally input from other imaging modalities, integrating OCT scan information and/or information from other modalities and/or recorded historical information, optionally some or all of the above with reference to a common coordinate system. Some embodiments comprise a display for displaying some or all of this information. In some embodiments, inferences based on observed portions of the organ relative to non-observed portions of an organ are displayed.

U.S. Pat. No. 7,952,718 discloses that “Mechanically robust minimal form factor OCT probes suitable for medical applications such as needle biopsy, intraluminal and intravascular imaging are achieved in part by employing compound lenses with some or all of the optical elements, including an optical fiber, to be thermally fused in tandem. To achieve a desired working distance without increasing a diameter of the optics assembly, a spacer can be disposed between the optical fiber and focusing optics. The compound lens configuration can achieve higher transverse resolution compared to a single lens at a desired working distance without increasing the probe diameter. In exemplary needle biopsy embodiments, the optical assembly is encapsulated in a glass housing or metal-like housing with a glass window, which is then selectively passed through a hollow needle. Esophageal imaging embodiments are combined with a balloon catheter. Circumferential and three-dimensional spiral scanning can be achieved in each embodiment.

U.S. Pat. No. 7,952,718 discloses “An apparatus for needle biopsy with real time tissue differentiation using one dimensional interferometric ranging imaging, comprising a biopsy device having a barrel and a needle, an optical fiber inserted in the needle, and a fiber optic imaging system connected to the optical fiber. The imaging system obtains images and compares the optical properties and patterns to a database of normalized tissue sample images to determine different tissue types. The physician performing the biopsy obtains feedback via a feedback unit associated with the biopsy device and which is connected to the imaging system. The feedback unit can provide visual, audible or vibratory feedback as to tissue type encountered when the needle is inserted toward the target tissue. The feedback unit can be programmed for different biopsy procedures so that the user can actuate a button to select a display or other feedback mechanism for the desired procedure and anticipated tissue to be encountered.”

U.S. Pat. No. 6,564,087 discloses “A fiber optic needle probe for measuring or imaging the internal structure of a specimen includes a needle defining a bore, an optical fiber substantially positioned within the bore, and a beam director in optical communication with the optical fiber. At least a portion of the wall of the needle is capable of conveying light. The beam director directs light from the optical fiber to an internal structure being imaged and receives light from the structure through a transparent portion of the wall. An actuating device causes motion of any, or all of, the needle, optical fiber, and beam director to scan the internal structure of the specimen. The fiber optic needle probe allows imaging inside a solid tissue or organ without intraluminal insertion. When used in conjunction with an OCT imaging system, the fiber optic needle probe enables tomographic imaging of the microstructure of internal organs and tissues which were previously impossible to image in a living subject.”

### SUMMARY OF THE INVENTION

According to an aspect of some embodiments of the invention, there is provided an system for optical coherence tomography (OCT) system of tissue of a subject comprising: a biopsy access tube suitable for insertion into the tissue, the access tube including a cannula of between 7G to 25G with a proximal opening, a distal opening and a lumen linking the proximal opening to the distal opening; and an elongated tool including a light guide sized to fit through the proximal opening and the lumen and long enough to convey light from the distal opening to the proximal opening and a distal portion sized to pass through the proximal opening, the lumen and the distal opening into the tissue; the distal portion including a window and a cavity containing a light directing element; the light directing element rotating over an azimuthal angle of between 45 to 360 degrees; the light directing element redirecting a light beam entering the window to the light guide; the window including a transparent pane preventing contact between the light directing element and the tissue.

According to some embodiments of the invention, the cannula is rigid.

According to some embodiments of the invention, the tool is fully retractable from the access tube.

According to some embodiments of the invention, the system further comprises: a GRIN lens between the light directing element and the light guide.

According to some embodiments of the invention, the light guide includes an optical fiber.

According to some embodiments of the invention, the optical fiber is flexible.

According to some embodiments of the invention, the light guide and the light directing element are shaped and sized to pass through the lumen without interference.

According to some embodiments of the invention, the tool further comprises: a location indicator retractable from the access tube with the light directing element.

According to some embodiments of the invention, the location indicator includes a position sensor with at least 5 degrees of freedom.

According to some embodiments of the invention, the location indicator includes fiduciary marker.

According to some embodiments of the invention, the system further includes a processor to compute a location of a source of the light beam based on a location of the location indicator.

According to some embodiments of the invention, the tool further comprises: an interferometer receiving the light beam from a proximal end of the light guide.

According to some embodiments of the invention, the tool further comprises: a wireless transceiver for transmitting data from the interferometer to a processor.

According to some embodiments of the invention, the tool further comprises a power source mounted to the tool for powering the interferometer and the transceiver.

According to some embodiments of the invention, the tool further comprises: a rotational optical coupler conveying the light beam from the light guide to the interferometer.

According to some embodiments of the invention, the light guide and the light directing element match a cross section of the lumen.

According to an aspect of some embodiments of the invention, there is provided a biopsy access tube for OCT scanning comprising: a cannula of between 7G to 25G with a proximal opening, a distal opening; a lumen linking the proximal opening to the distal opening; a window in the cannula between the proximal opening and the distal opening the window directed over an azimuthal angle between 45 to 360 degrees; the window including a pane, the pane conveying infrared light between the lumen and an exterior of the cannula.

According to some embodiments of the invention, the lumen is straight.

According to some embodiments of the invention, the window includes a distal tip of the cannula.

According to some embodiments of the invention, the lumen has a minimal cross sectional area between the distal opening and the proximal opening ranging between 25% to 80% of an average cross sectional area of the cannula.

According to some embodiments of the invention, the distal opening has a minimal cross sectional area of at least 25% of an average cross sectional area of the cannula.

According to some embodiments of the invention, the biopsy access tube further comprises: a light directing element slidably fitting into the lumen from the proximal opening to the window, the light directing element redirecting a beam to a light guide leading to the proximal opening.

According to some embodiments of the invention, the light guide includes an optical fiber.

According to some embodiments of the invention, the biopsy access tube further comprises: a rotational controller directing an azimuthal angle of the light directing element from outside the biopsy access tube.

According to some embodiments of the invention, the biopsy access tube further comprises: a plug mounted distally to the light directing element, the plug sliding axially with the cannula, the plug blocking fluid communication between the distal opening and the light directing element.

According to some embodiments of the invention, the pane, the cannula and the plug prevent fluid communication between the light directing element and an exterior of the cannula.

According to some embodiments of the invention, the biopsy access tube further comprises: a rotational connector between the light directing element and the plug allowing the plug to rotate around an axis of the cannula independently of the plug.

According to some embodiments of the invention, the pane is composed of glass.

According to some embodiments of the invention, the pane is composed of Polycarbonate.

According to some embodiments of the invention, the pane is composed of sapphire.

According to an aspect of some embodiments of the invention, there is provided an scanning OCT system comprising: a biopsy access tube including rigid cannula of between 7G to 25G including a lumen with a proximal opening; a tool including a light guide sized to fit into the lumen of the biopsy access tube, a scanning head sized to fit within the lumen; the scanning head light attached to a distal end of the light guide; the scanning head directing a light beam from outside the access tube to the light path, the scanning head being adjustable to redirect light from different azimuthal angles with respect to the access tube and a location indicator having a fixed spatial orientation with respect to the scanning head and retractable from the access tube with the scanning head.

According to some embodiments of the invention, the tool is fully retractable from the access tube.

According to some embodiments of the invention, the system further comprises: a GRIN lens between the light scanning head and the light guide.

According to some embodiments of the invention, the system further comprises: an interferometer permanently fixed in orientation with respect to a proximal end of the light guide.

According to some embodiments of the invention, the system further comprises: an OCT engine receiving the light from a proximal end of the light guide; a processor for computing a 3D image map from the OCT engine and the location indicator.

According to some embodiments of the invention, the system further includes: a wireless transceiver for conveying data from the OCT to the processor.

According to some embodiments of the invention, the system further comprises: a rotational optical coupler conveying the light from the light guide to the OCT engine.

According to some embodiments of the invention, the location indicator retractable from the access tube with the light directing element.

According to some embodiments of the invention, the location indicator includes a position sensor with at least 5 degrees of freedom.

According to some embodiments of the invention, the location indicator includes fiduciary marker.

According to some embodiments of the invention, the system further includes a processor to compute a location of a source of the light based on a location of the location indicator.

According to an aspect of some embodiments of the invention, there is provided a method for directing a procedure in a region of interest (ROI): inserting an access tube to the ROI; introducing a OCT scanning head into the ROI via a lumen of the access tube; scanning the ROI with the scanning head while located in the ROI; retracting the scanning head from ROI via the access tube while the access tube remains in the ROI; introducing a second tool to the ROI via the access tube; performing a second procedure with the second tool.

According to some embodiments of the invention, the introducing a second tool is subsequent to the retracting and wherein the scanning includes determining a location for the second procedure.

According to some embodiments of the invention, the introducing a second tool is prior to the introducing the OCT scanning head and wherein the scanning includes evaluating the second procedure.

According to some embodiments of the invention, the method further includes: indicating a 5 DOF location of the scanning head to determine a 3D location of the second procedure.

According to some embodiments of the invention, the second procedure includes ablating the lesion.

According to some embodiments of the invention, the second procedure includes ablating lesion and wherein the evaluating includes determining a completeness of the ablating.

According to some embodiments of the invention, the method further includes: building a composite 3D image based on the scanning.

According to some embodiments of the invention, the access tube had an outer diameter 7G to 25G.

According to some embodiments of the invention, the access tube is rigid.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart illustration of a method of scanning a region with an OCT scanner introduced into a multifunction OCT biopsy access tube in accordance with an embodiment of the present invention;

FIG. 2 is a flow chart illustration of a method of scanning a region with an OCT scanning tool introduced into a biopsy access tube in accordance with an embodiment of the present invention;

FIG. 3 is a block diagram illustrating a multifunction OCT biopsy access tube in accordance with an embodiment of the present invention;

FIG. 4 is a is a block diagram illustrating an OCT scanning tool introducible into a standard biopsy access tube in accordance with an embodiment of the present invention;

FIG. 5 is a high level block diagram illustrating optional geometries for placement of components of the OCT engine and other subsystems in a OCT scanning system in accordance with some embodiments of the present invention;

FIG. 6 is a flow chart illustration of various optional steps in a method of real time control and/or review of an internal procedure using an OCT tool in accordance with an embodiment of the present invention;

FIG. 7 is a cross sectional view of an OCT biopsy system including a multifunction access tube with a window near the distal end in accordance with an embodiment of the present invention;

FIG. 8 is a is a cross sectional view of an OCT biopsy system including a multifunction access tube with a window at the distal end in accordance with an embodiment of the present invention;

FIG. 9A is a cross sectional view of a multifunction access tube being used to place a fiducial marker in accordance with an embodiment of the present invention;

FIG. 9B is a cross sectional view of a multifunction access tube with a distal location indicator in accordance with an embodiment of the present invention;

FIG. 9C is a cross sectional view of a multifunction access tube being used to take a conventional biopsy in accordance with an embodiment of the present invention;

FIG. 9D is a cross sectional view of a multifunction access tube with a core needle in accordance with an embodiment of the present invention;

FIG. 10A is a cross sectional view of a conventional access tube and an OCT tool head in accordance with an embodiment of the present invention;

FIG. 10B is a cross sectional view of a conventional access tube being used to position an OCT tool head in accordance with an embodiment of the present invention;

FIG. 10C is a cross sectional view of an OCT tool head exposed to tissue in accordance with an embodiment of the present invention;

FIG. 11A is a cross sectional view of a conventional access tube being and an OCT tool head having a obturator tip in accordance with an embodiment of the present invention;

FIG. 11B is a cross sectional view of an OCT tool head with a obturator tip exposed to tissue in accordance with an embodiment of the present invention;

FIG. 12 is a cross sectional view of an OCT tool with a non-rotating with a obturator tip in a multifunction OCT access tube in accordance with an embodiment of the present invention;

FIG. 13 is a cross sectional view of an OCT access tube with a distal scanning motor in accordance with an embodiment of the present invention;

FIG. 14 is a cross sectional view of an OCT tool and handset in accordance with an embodiment of the present invention;

FIG. 15 is a cross sectional view of an OCT access tube and OCT tool handset including a localization sensor in accordance with an embodiment of the present invention;

FIG. 16A-C is a cross sectional view of an OCT tool handset including a localization sensor and a rotational coupler in accordance with an embodiment of the present invention;

FIG. 17 is a cross sectional view of an OCT tool handset including a localization sensor, a rotational coupler and an interferometer in accordance with an embodiment of the present invention;

FIG. 18 is a cross sectional view of an OCT tool handset including a localization sensor, a rotational coupler, an interferometer and a light source in accordance with an embodiment of the present invention, and

FIG. 19 is a cross sectional view of a wireless OCT tool handset including a localization sensor, a rotational coupler, an interferometer and a light source, a power source and a wireless transmitter in accordance with an embodiment of the present invention.

### DESCRIPTION OF SPECIFIC EMBODIMENTS OF THE INVENTION

The present invention, in some embodiments thereof, relates to a biopsy access tube, tools and/or methods, and, more particularly, but not exclusively, to tools and/or methods for sharing a access tube for multi-dimensional Optical Coherence Tomography scanning and other procedures.

### Overview

Various aspects of the current invention relate to methods and/or systems to for multidimensional and/or composite OCT imaging and/or facilitate use of OCT together with other technologies (for example biopsy technologies and/or therapeutic or diagnostic technologies). In some embodiments OCT tools facilitate and/or improve OCT diagnostic procedures. In some embodiments tools and/or subassemblies facilitate use of OCT in real time therapeutic procedures. For example, a multi-use OCT/Biopsy access tube and/or tool facilitate use of OCT imaging in conjunction with other biopsy tools.

In some embodiments, Optical Coherence Tomography may refer to use of light to produce an image that shows internal detail structures and/or tissue in a region of interest (ROI). Optionally OCT may be combined with scanning. For example scanning may include making a panoramic image and/or an image with a moving scanning head and/or combining OCT images at known locations to produce a composite image and/or collage image of a multidimensional region (for example a 2D area and/or a 3D region). Optionally OCT images may be combined with images made using other technologies.

In some embodiments, scanning may include 3D mapping. For example 3D mapping may include building a 3D model of the ROI, tracking the location of a location indicator and/or the body of a subject and/or correlating the data to relate a current OCT image to previous images, images produced by other probes, images produced by other methods and/or features in the body of the subject.

In some embodiments, the tools disclosed herein may be used for optical tools and/or methodologies other than OCT.

For simplicity of exposition, electromagnetic waves used in OCT will sometimes be referred to herein as “light”, but it is to be understood that wavelengths including visible light, Near-IR wavelengths and/or other IR wavelengths are also being referred to in references herein to “light” used in OCT.

An aspect of some embodiments of the present invention relates to the use of OCT for real time control and/or evaluation of a medical procedure. In some embodiments of the present invention a single lumen may be used to facilitate OCT scanning and/or therapeutic access to nearby and/or overlapping regions. Optionally, OCT imaging and/or OCT scanning and/or other diagnostic procedures and/or other detection procedures and/or therapeutic procedures may share a single access tube and/or a single lumen of a multi-lumen for sequential and/or repeated and/or simultaneous access to a region of interest ROI. Optionally facilitating multiple and/or coordinated access may allow improved control and/or evaluation of medical procedures. Control and/or evaluation is optionally in real time and/or during a procedure.

Some embodiments of the present invention expand the scanning ability of OCT systems by providing means and/or methods for combining scan information from a plurality of access tubes and/or from a plurality of tissue insertions of same access tube, recording that information in a common unified three-dimensional coordinate system. The tools and/or methodology may facilitate scanning and/or recording information from a tissue volume larger than that which can be scanned by a single access. OCT systems utilizing some embodiments of the present invention may be used to combine, coordinate, and/or collectively analyze information gleaned from OCT scans performed at a plurality of positions and/or during a plurality of “tissue insertions” (insertion of access tube into tissue for scanning purposes). This plurality of tissue insertions may be performed by one access tube in a plurality of sequential insertions, and/or by (optionally simultaneous) insertions of a plurality of access tubes into tissue. Both methods may be used to use OCT access tubes to scan a large tissue volume. In this manner, in some embodiments, an entire organ, such as for example a prostate, can be scanned in sufficient detail to detect clinically significant tumors or other lesions.

A detailed three-dimensional mapping and/or modeling of the lesion, optionally obtained from a plurality of access tube insertions into a lesion and/or into tissue around a lesion may provide a detailed guide for a surgical procedure. Alternatively, such a map and/or model may provide means for a series of detailed anatomical comparisons of views of a ROI region, taken over time.

Some embodiments of the present invention may include one or more localization indicators. For example, a localization indicator may include a position and/or orientation sensor. Alternatively or additionally, a localization indicator may include a marker (for example a marker that can be seen using fluoroscopy and/or other imaging technology). In some embodiments an indicator may include a relative position indicator. For example, an indicator may include a radio indicator for a local positioning system and/or a beacon and/or an indicator of relative position between an access tube and/or a tool. For example, a window may have markers that allow a user to determine where an OCT image is with respect to the position of an access tube and/or the access tube may include a localization indicator (for example the indicator may have 1, 2, 3, 4, 5, or 6 degrees of freedom). Optionally, a tool sized and shaped to fit into the access tube may have an indicator of position relative to the access tube (for example length of insertion and/or relative rotational orientation) and/or a tool may have an indicator of position relative to a local object (for example a marker in the body of a patient and/or a fixed marker in an operating theater). Optionally an access tube and/or tool may include a position indicator on a distal location (for example at the tool location in the patient) and/or at a proximal location (for example in a handset and/or handle of the tool and/or access tube). For example a position indicator may include a five or six degrees of freedom (DOF) assembly sensor. For example a location sensor may include sensor models 55, 90, 130, 180 and/or 800 sensor available from Ascension Technology Corporation, 6221 Shelburne Road, Suite 130, Shelburne, Vt. 05482, USA. Alternatively or additionally, a position indicator may include a fiducial marker for example an implant such as FlexiCoil™, PolyMark™ or Gold Soft Tissue markers, available from Civco 2301 Jones Blvd, Coralville, Iowa 52241.

An aspect some embodiments of the current invention relates to a multifunction biopsy system including a access tube and/or probe and/or OCT tool with a side looking light conveying window and/or an OCT scanning head sized and shaped to fit through the access tube and/or direct light through the window. Optionally, the OCT scanning tool may scan azimuthally while the tool and/or window remain stationary and/or are translated longitudinally. A system, including the access tube (for example including a standard biopsy access tube) and/or OCT tool, optionally includes a localization assembly that allows an automated system to the exact location of the OCT scan and/or other procedures. In some embodiments, the OCT tool may be introduced and/or fully retracted to and/or from the access tube.

In some embodiments, the access tube is inserted into the diagnosed tissue with a core needle. After insertion, the core needle is optionally retracted and/or the OCT tool is introduced into the lumen of the access tube. Alternatively or additionally, an OCT tool may include a obturator. For example, the biopsy access tube could be inserted into a tissue with the OCT tool in the lumen of the access tube.

In some embodiments, in order to examine tissue, the OCT tool may be exposed to the tissue by being pushed forward out of the access tube into the tissue. Alternatively or additionally, the access tube may be retracted backwards exposing the OCT tool to the tissue. Optionally the tool may include a scan head. For example the scan head may be rotated around the axis of the access tube for example to scan a wedge and/or a disk shaped region and/or radial sections of a disk shaped region. Alternatively or additionally, the scan head may be moved along the axis of the access tube for example to scan a linear region. Alternatively or additionally the scan head may be moved linearly and rotated for example to scan a helical region and/or a series of wedge and/or a disk shaped regions.

In some embodiments, an axial beam is redirected by scan head through the window. In some embodiments, a reflected beam is redirected by a scan head along a light guide to a detector. Optionally the light guide may pass along the lumen of an access tube. The light guide may include, for example a GRIN (Gradient Index Lens) and/or an optical fiber OF.

In some embodiments, the OCT tool is fully retracted out of the access tube. For example, after retracting the OCT tool (and/or before introducing it) a different diagnostic tool and/or treatment tool may be introduced into a ROI scanned by the OCT tool and/or into a neighboring area. OCT may optionally be used to detect and/or identify a structure and/or lesion. Optionally, a sampling tool and/or an intervention tool may be introduced to get a tissue sample from and/or to treat the OCT scanned ROI and/or another location. For example, an ablation tool may be used to treat and/or destroy a structure identified by the OCT scan. The OCT tool may further be used to evaluate whether the sampling and/or intervention were successful and/or properly focused and/or complete. In some cases, further intervention can be applied. For example, the OCT tool can be retracted and/or the access tube may remain in place to guide further tools for further evaluations and/or interventions.

An aspect some embodiments of the current invention relates to a multipurpose access tube. Optionally the multipurpose access tube includes a side looking light conveying window near a distal opening of the access tube. Optionally, the distal opening may be on an end of the access tube and/or in a side of a distal portion of the access tube. For example, the access tube may be used with an OCT scanning tool that directs light through the window. Optionally, an OCT scanning head may be configured to scan azimuthally while the access tube and/or window remain stationary and/or are translated longitudinally. For example the scanning head may be shaped to rotate behind the window and/or inside the lumen of the access tube. A system, including the access tube and/or OCT tool, optionally includes a localization assembly that allows an automated system to track the location of the OCT scan and/or other procedures.

In some embodiments, the access tube may be used with internal interchangeable tools. Optionally, with a single puncture of the patient body multiple tools may reach a target organ without the need for re-insertion of the needle more than once to that location. Optionally, introducing multiple tools with a single access tube and/or a single lumen of a multi-lumen access tube reduces undesired effects such as bleeding, contamination, infections etc.

In some embodiments, a window may cover for example an azimuthal region of between 30 to 120 degrees and/or between 120 to 180 degrees and/or between 180 to 270 degrees and/or between 270 to 360 degrees. The OCT scan head may optionally be mounted on a rotating assembly to scan across the azimuthal angle of the window. Alternatively or additionally, The OCT scan head may optionally be mounted on a longitudinally moving assembly for moving the scan head longitudinally along the needle. The distal opening of an access tube may for example be used for introduction of conventional and/or specialized tools.

In some embodiment an access tube may include a cannula of diameter ranging between 7 to 14G and/or between 14 to 18G and/or between 18 to 22G and/or between 22 to 30G. In some embodiments the cannula may have a lumen having a cross sectional area ranging between 2 to 10% and/or between 10 to 30% and/or between 30 to 50% and/or between 50 to 75% and/or between 75 to 90% and/or between 90 to 100% of the entire cross sectional area of the cannula. In some embodiments the proximal and/or distal openings of the cannula may have a cross sectional area ranging between 2 to 10% and/or between 10 to 30% and/or between 30 to 50% and/or between 50 to 75% and/or between 75 to 90% and/or between 90 to 100% and/or between 100 to 200% or more of the cross sectional area of the cannula lumen.

In some embodiments, the access tube includes a needle and/or a cannula. The cannula optionally has a sharp edge on its distal side to cut its way into a patient for example to reach a target tissue. In some embodiments, a window may include the sharp edge. For example the window may be made of a hard material, for example sapphire, quartz or a polymer. Alternatively or additionally the distal end of the access tube may include an extension for cutting into tissue, for example a metal tip and/or blade. Optionally, the cannula may be made of stainless steel or hard plastic material.

In some embodiments, a thin polymer film, for example Parylene, is deposited on the outer surface of the cannula. For example the film may protect the patient in the case of breakage. Optionally, parts of the system, for example the cannula and/or the tool may be made of metal and/or polymers and/or may be further coated or treated. For example all or part of an outer surface may be coated, for example with Teflon or Molybdenum di-Sulfide in order to reduce friction. Other coatings are optionally included.

In some embodiments, the access tube may include a proximal port similar to a conventional biopsy access tube. The proximal port optionally accepts various introducible internal tools sequentially and/or simultaneously.

In some embodiments, the OCT tool and/or the access tube may be connected to a localization sensor and/or a handset for controlling the OCT tool and/or scanning head and/or for indicating the location of the OCT tool and/or scanning head for example as described in hereinbelow.

Some embodiments an access tube may include a cannula, (for example a conventional biopsy access tube cannula) which may have for example an outer diameter (OD) ranging for example between 0.2 to 0.8 mm and/or between 0.8 to 1.6 mm and/or between 1.6 to 3 mm. The cannula may have an inner diameter (ID) ranging between 80% to 90% the OD and/or between 50% to 80% the OD and/or between 90% to 99% the OD. A biopsy and/or OCT tool may in some embodiments be sized and shaped to fit into the cannula. For example the tool may have an outer diameter (OD) substantially equal to the inner diameter of the cannula. For example the OD of the tool may range between 98-100% the ID of the cannula and/or between 95-98% the ID of the cannula and/or between 90-95% the ID of the cannula and/or between 80-90% the ID of the cannula and/or between 60-80% the ID of the cannula.

In some embodiment a window may be an open aperture and/or may include a pane conveying light over a desired frequency range. For example a pane may be made of a suitable polymer, glass and/or other light conveying material at the desired wavelength (for example 0.8-1.7 Microns) known in the art (for example Sapphire, Polycarbonate, Silicon, Zinc Sulfide, Borosilicate and/or Germanium etc.).

In some embodiments a biopsy needle may be rigid, for example a 10 cm rigid needle may deflect less than 1 mm when or between 1 to 10 mm and/or between 10 to 20 mm when a 100 gram force is applied to an end of the needle perpendicular to its axis. In some embodiments a rigid needle may have a maximum elastic bend of less than 5 degrees and/or between 5 and 10 degrees and/or between 10 to 20 degrees and/or between 20 to 50 degrees.

In some embodiments a biopsy needle may be flexible, for example a 10 cm flexible may deflect between 20 to 50 mm when a 100 gram force is applied to an end of the needle perpendicular to its axis. In some embodiments a flexible needle may have a maximum elastic bend of greater than 20 degrees and/or between 20 and 50 degrees and/or between 50 to 90 degrees and/or greater than 90 degrees. In some embodiments a flexible cannula may be inserted with a stylet.

In some embodiments some and/or all parts of an OCT tool may be connected to a stationary consol, the access tube, the tool and/or a handset of the tool and/or access tube. For example, a consol may be connected to the tool via a wire and/or an optical fiber and/or a wireless channel. For example, an OCT system may include a localization sensor, an optical coupler (for example a rotating optical coupler), an actuator (for example a rotary actuator to rotate a scanning head and/or a linear actuator to position a sensor along a cannula), an illumination source, an interferometer, a radiation detector, a wired and/or wireless communication transceiver and/or a power source. One some and/or all of the component of the system may be mounted to a access tube and/or a tool sized and shaped to fit in the access tube and/or a handset of the tool and/or on an external consol. For example an illumination source and/or detector may be mounted in a consol and/or connected to a tool via an optical fiber for example reducing the number of components on the tool. Alternatively or additionally, the detector and/or illumination source may be located in the tool and/or access tube. Power may be supplied to the tool and/or access tube via wires and/or communication between the tool and/or a consol may be via a hardwired connection, for example increasing maneuverability of the tool. Alternatively or additionally, the tool may include a power source and/or a wireless transceiver, for example for un-tethered use.

### DETAILED EMBODIMENTS

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

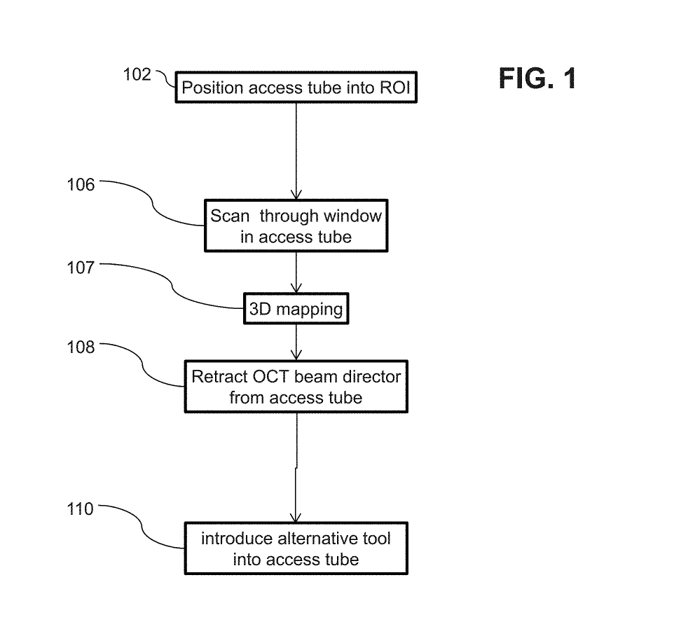

Referring now to the drawings, FIG. 1 is a flow chart illustration of a method of scanning a region with an OCT scanner introduced into a multifunction OCT biopsy access tube in accordance with an embodiment of the present invention. In the example a access tube having a distal window and/or a distal opening is inserted 102 into a region of interest (ROI). An OCT tool is sized and shaped to fit into the access tube and is positioned and/or moved inside the access tube to scan 106 the ROI through the window. Scanning 106 may optionally include 3D mapping 107 of the image and/or features. Optionally when access tube is not being used for OCT scanning, the OCT tool is retracted 108 and/or a different tool is introduced 110 into the access tube. For example a different tool may include a diagnostic tool (for example a sample taking tool and/or a thermometer and/or an Infrared camera) and/or a treatment tool (for example an ablation tool and/or an aspiration tool and/or ultrasonic device and/or a cryoaccess tube and/or a brechytherapy device). Alternatively or additionally, the access tube may be employed first to introduce a non-OCT tool and then the OCT tool may be introduced later. For example the OCT tool may be used to evaluate an effect of the non-OCT tool.

In some embodiments the window may include be a 360 degree wrap around azimuthal pane capable of conveying light of a desired wavelength to a sufficient degree (e.g. transparent). Alternatively or additionally the window may include a pane covering an azimuthal angle of between 45 to 360 degrees. Alternatively, the window may consist of multiple panes arranged at various locations and/or azimuthal angles. For example, a window pane may include be made of Glass, quartz, sapphire, spinel, polycarbonate and/or other known IR conveying polymers. Optionally the pane may convey light of a wavelength range for example between 700 to 1700 micrometers or a portion thereof and/or between 400 to 600 micrometers and/or between 600 to 800 micrometers and/or between 800 to 1000 micrometers and/or between 1000 to 1300 micrometers and/or between 1300 to 1700 micrometers and/or between 1700 to 2000 micrometers and/or between 2000 to 5000 micrometers. Light of the preferred band may be conveyed with an attenuation ranging for example between 10 dB/km to 60 dB/km and/or between 60 dB/km to 120 dB/km and/or between 120 dB/km to 400 dB/km and/or between 400 dB/km to 1000 dB/km. The thickness of a pane may range for example between 0.02 mm to 0.05 mm and/or between 0.05 mm to 0.1 mm and/or between 0.1 mm to 0.3 mm and/or between 0.3 mm to 1.0 mm. For example, between 10 to 30% and/or between 30 to 50% and/or between 50 to 70% and/or between 70 to 90% and/or between 90 to 99% of light of the preferred band that is directed at the pane may be conveyed across the pane. Optionally the window may include a series of holes in the side of the access tube without a light conveying pane. Optionally the window may include markers that indicate the angular relation between the OCT scanner and/or the access tube. For example, the window may have an opaque section such that as a scanner moves, as it passes the opaque section wherein the OCT signal is blocked. Optionally from the output of the scanner the times when it is blocked are known and the position and/or orientation and/or scanning rate of the scanner can be derived.

In some embodiments, scanning may include rotating a scanning head to achieve a azimuthal scan (for example of a disk shaped area) and/or moving a scanning head longitudinally along a cannula and/or window of the access tube to make a linear scan and/or changing an attitudinal angle (the angle with respect to the axis of the access tube). Optionally linear and/or azimuthal movement may be used to make a helical scan and/or azimuthal and/or attitudinal movement may be used to make a spherical scan. Optionally there may be multiple windows and/or one long window along the length of the access tube for scanning at different longitudinal locations.

In some embodiments the access tube may be inserted into a ROI with the OCT tool in its lumen (for example the OCT tool may include a obturator tip that is sized and shaped to fit through the distal opening of the tool). Alternatively or additionally, the OCT tool may be introduced after positioning of the access tube. In some embodiments, an OCT tool may be introduced into the access tube after the access tube has been used for performing a different procedure. Optionally in some embodiments various tools and/or an OCT access tube may be introduced into the access tube and/or used without moving the access tube. For example, multiple tools may be used to treat and/or scan the same region with multiple procedures and/or without requiring multiple puncturing of the patent and/or ROI. Examples of access tubes and/or tools that may be used with an OCT system and/or method as described herein include Standard core biopsy assembly or FNA biopsy assembly, for example C.R. Bard Magnum series needles: Bard Biopsy Systems—A Business Unit of Bard Peripheral Vascular, Inc. 1415 West Third Street, Tempe, Arizona 85281, USA, www.bardbiopsy.com and/or a cryoaccess tube for example available from Galil Medical cryo-access tubes: Galil Medical Inc., 4364 Round Lake Road, Arden Hills, Minn. 55112 USA; and/or Brechytherapy tools and/or needles for example FASTFILL Seed implant needle, available from Bard Medical Division, 8195 Industrial Boulevard, Covington, Ga. 30014.

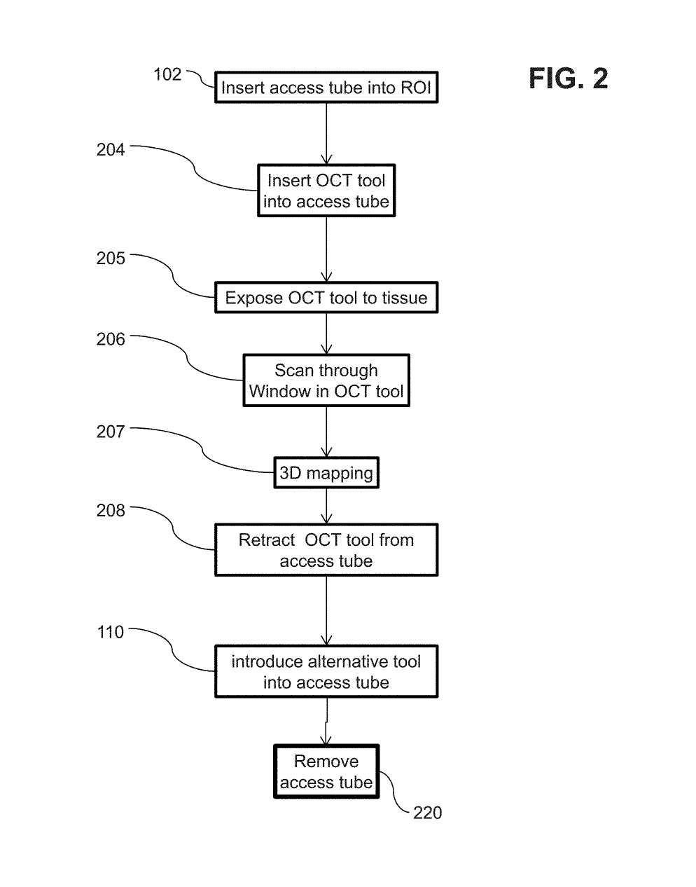

FIG. 2 is a flow chart illustration of a method of scanning a region with an OCT scanning tool introduced through a biopsy access tube in accordance with an embodiment of the present invention. In some embodiments the OCT scanning tool may have a scanning head and/or a light conveying window (for example a light conveying tube and/or a window pane). The scan head may directs light through the window. Optionally the tool includes a mechanism to move the scan head (for example longitudinally and/or azimuthally. The tool may be introduced through an access tube and/or out a distal opening of the tube into the region of interest. For example via this methodology OCT scanning tool may be introduced using a standard biopsy access tube.

In some embodiments, a standard biopsy access tube with a distal opening and/or a proximal opening is inserted 102 into a region of interest. An OCT scanning tool is optionally introduced 204 into the access tube before or after insertion into the ROI (for example the OCT may be connected to a obturator tip and/or inserted with the access tube and/or the access tube may be inserted with a separate obturator tip and then the obturator tip removed and/or an OCT tool introduced into the access tube). Following insertion, the OCT tool is optionally exposed 205 to tissue in the ROI. For example the tool may be pushed out the distal opening of the access tube and/or the access tube may be retracted leaving the tool in the ROI.

In some embodiments, an OCT scanning head (for example a beam director) may be located inside a light conveying shield on the OCT tool. For example, the shield may be exposed to the tissue and/or remain stationary while the scanning head moves inside the tool to scan 206 the ROI. Scanning 206 may optionally include 3D mapping 207 of the image and/or features.

In some embodiments, when scanning 206 is completed, the OCT tool may be retracted 208 from the ROI and/or through access tube to outside the access tube and/or subject. The access tube may optionally be used to introduce 110 another tool (before or after scanning 206). Optionally, multiple procedures may be performed through a single access tube and/or a single lumen after a single insertion into the patient. In some embodiments, after one or more procedures, the access tube may be removed 220 from the patient and/or moved to another location.

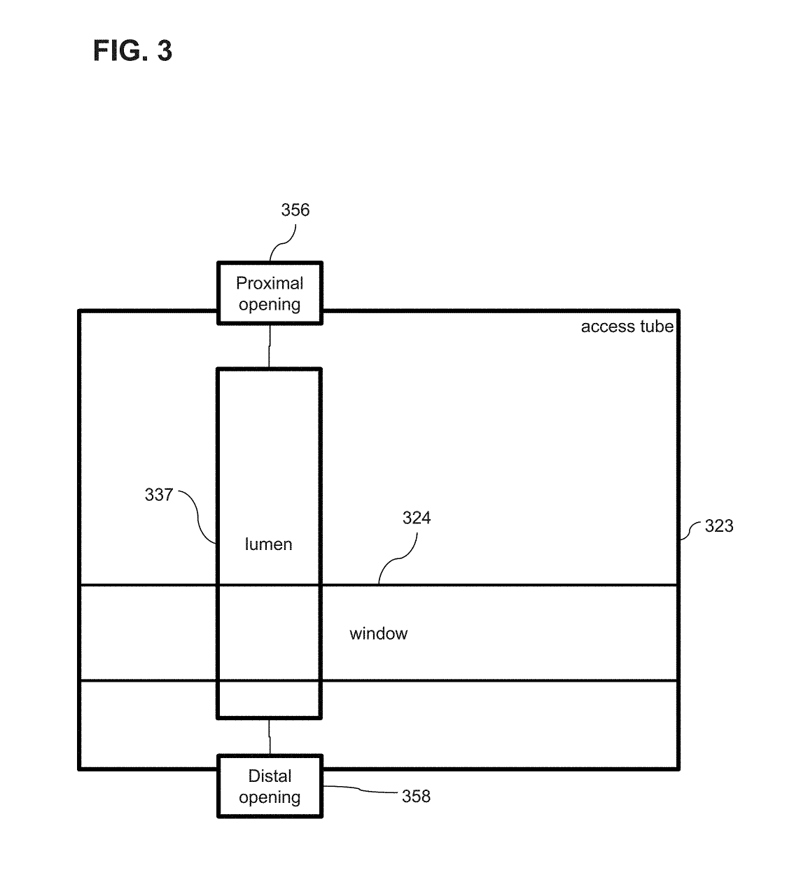

FIG. 3 is a block diagram illustrating a multifunction OCT biopsy access tube 323 in accordance with an embodiment of the present invention. In some embodiments, a biopsy access tube will include a lumen 337 with a proximal opening 356 and/or a distal opening 358. For example, when the access tube 323 has been introduced into a subject, a tool may be introduced into access tube 323 through proximal opening 356 and/or out access tube 323 into a RIO through distal opening 358. Access tube 323 optionally further includes a window 324 located between proximal opening 356 and distal opening 358. For example an OCT scanning head may be introduced through the proximal 356 opening into lumen 337 and positioned on the inside of window 324 and/or to direct light through window 324. Optionally, the scan head can move inside lumen 337 to scan a ROI through window 324 (and/or through multiple locations and/or windows along and/or around lumen 337).

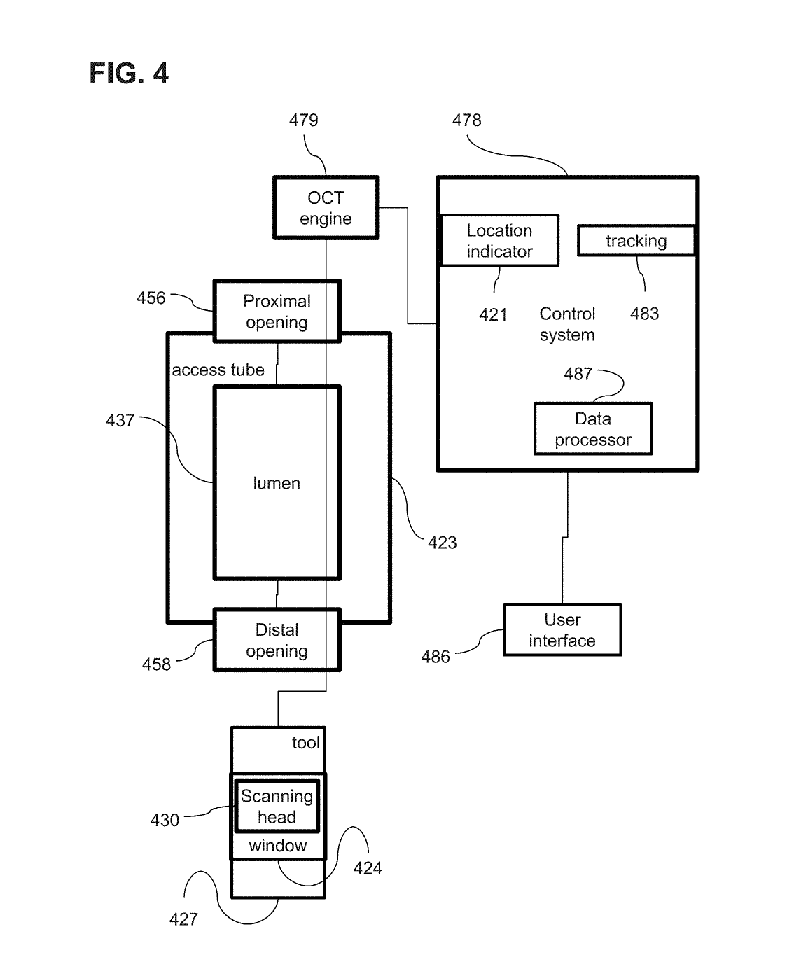

FIG. 4 is a is a block diagram illustrating an OCT scanning tool introducible into a biopsy access tube in accordance with an embodiment of the present invention. For example a tool 427 may be introduced into a ROI through a standard biopsy access tube 423. Optionally tool 427 includes a window 424. An optional scanning head 430 may be located inside the tool and/or shielded from contact with tissue by the window. Scanning head 430 is optionally configured to direct an OCT beam through window 424. Optionally scanning head 430 can redirect the beam to scan different portions of the ROI while window 424 remains stationary.

In some embodiments, tool 427 is introduced into a proximal opening 456 of the access tube and/or through a lumen 437 of the access tube and/or out a distal opening 458 of the access tube into a ROI (For example as illustrated in FIGS. 8-11 and 14). Window 424 may be exposed to tissue in the ROI and/or scan head 430 may be shielded from the tissue by a pane of window 424. A control system 478 and/or an OCT engine 479 of tool 427 may be used to control the scanning and/or determine the location being imaged and/or collect data from tool 427.

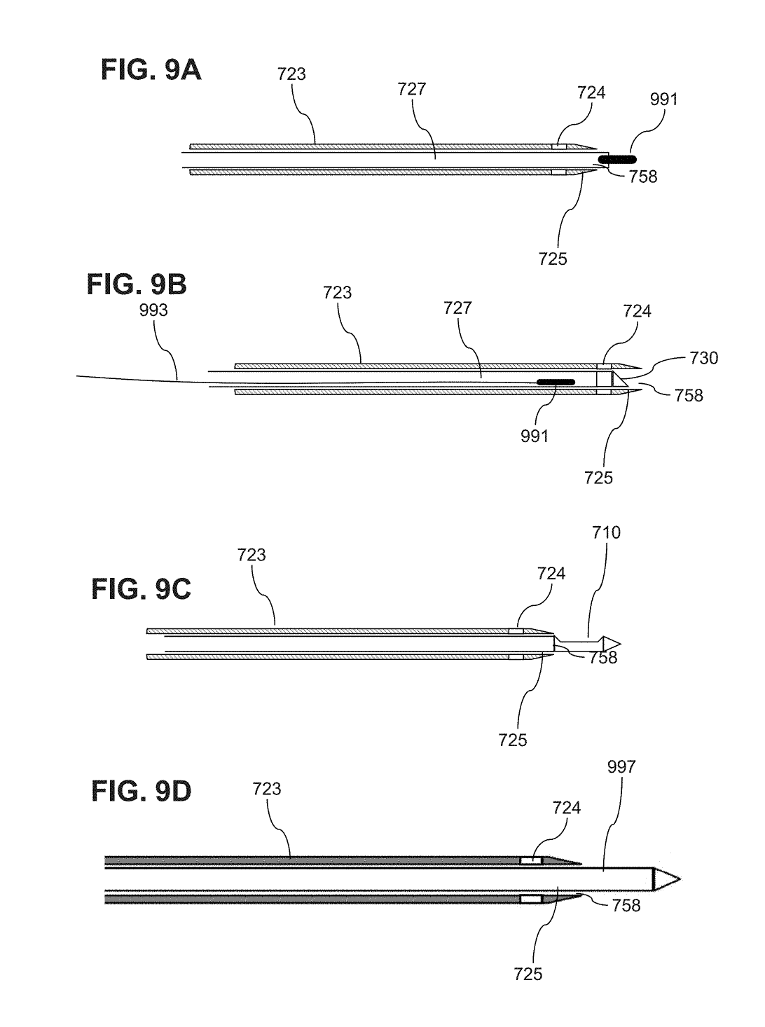

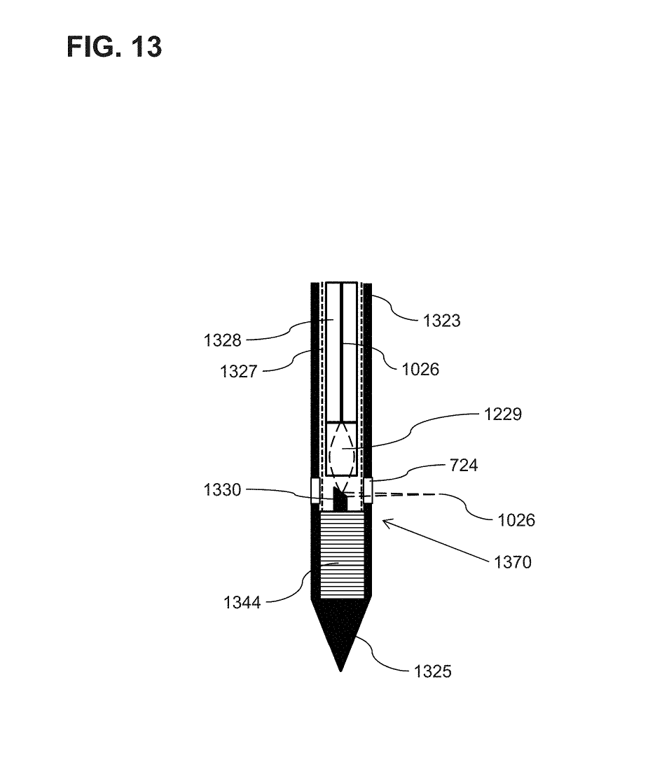

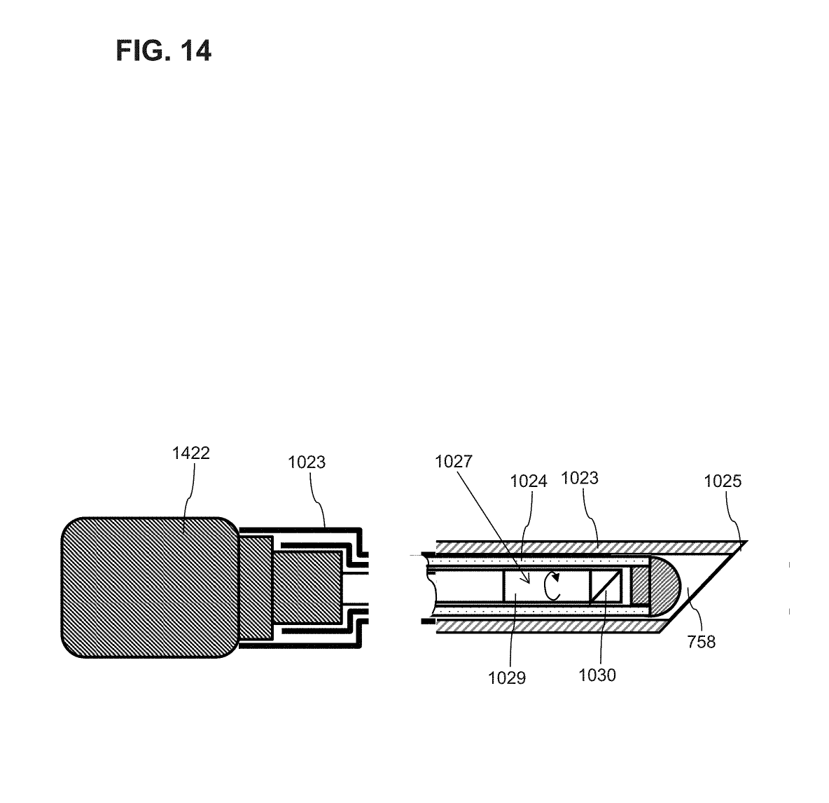

In some embodiments, all or part of control system 478 and/or OCT engine 479 may be housed in a handset of tool 427 (For example as illustrated in FIGS. 14-19). Optionally the handset may remain outside of access tube 423 and/or the subject. Alternatively or additionally, all or part of control system 478 and/or OCT engine 479 may pass into and/or through access tube 423 (for example as illustrated in FIG. 9B). Alternatively or additionally, all or part of control system 478 and/or OCT engine 479 may be housed in an external consol. Alternatively or additionally, all or part of control system 478 and/or OCT engine 479 may be housed in access tube 423 (for example as illustrated in FIG. 13).

In some embodiments, an OCT engine 479 may include an interferometer and/or a power supply and/or an illumination source and/or a rotating optical coupler (For example as illustrated in FIGS. 13-19). Optionally, the OCT tool and/or all or part of control system 478 and/or of an OCT engine 479 may be fully retractable from the access tube.

In some embodiments, control system 478 may include a communication means (for example a wired connection and/or a wireless transceiver) and/or a rotational indicator (indicating the rotation of the scanning head with respect to a reference for example the access tube) and/or a localization indicator (for example a 5 or 6 dimensional orientation sensor) and/or an actuator for controlling the orientation and/or position scan head 430 (For example as illustrated in FIGS. 13-19).

In some embodiments, the position of a location indicator 421 is tracked by a tracking module 483 which produces a location data stream. In some embodiments, an OCT engine produces an image data stream. Both data streams are optionally reported to a processor 487. Optionally, based on a known relationship between the position of location indicator 421 and the focus of scan head 430, processor 487 calculates positions of features of the imaged tissue, stores data in a memory, interprets and analyses, compares with historical data, and/or maps data in 3D. Processor 487 optionally correlates location data with data on location of a body of a subject. For example, subject location may include location of the L5 vertebra, whose movements may in some cases correlate with movements of the prostate. Optionally, processor 487 may correlate image location data with the location of the prostate. User interface 486 optionally includes hardware and software for organizing and communicating images of the features in views that are understandable according to a user and/or based on a user defined viewpoint and/or optionally displayed in real time display.

In some embodiments, location indicator 421 comprises, optionally a 5 or 6 degrees of freedom sensor. Location indicator 421 optionally detects and reports its own positions and orientations to location tracking module 483. Alternatively or additionally location indicator 421 comprises a fiducial marker and/or tracking module 483 includes a sensor (for example a fluoroscopic system and magnetic resonance system and/or an ultrasound system) to recognize the position and/or orientation of the fiducial marker. Alternatively or additionally, an initial position of scanning head 430 may be known and location indicator 421 may include an accelerometer to reporting changes of location which may be tracked by tracking module 483. Alternatively or additionally, templates or other forms of probe guides may be used to constrain movements of scanning head 430. In such a case, scanning head 430 location tracking may be highly simplified or unnecessary, since location information might then be known in advance and available to processor 487 for calculations. Examples of commercial systems that could serve as location tracking module 483 include electromagnetic tracking (e.g. Ascension Technology corp. Burlington, Vt., USA, and NDI's Aurora tracking system, Waterloo, Ontario, Canada), electromechanical tracking (cfEigen LLC., Calif, USA, Biobot Pte Ltd., Singapore), optical tracking (e.g. NDI, Polaris tracking system, Waterloo, Ontario, Canada), IR tracking, 4D Ultrasound tracking (e.g. GE Ultrasound, USA, Koelis, La Tranche, France), gyroscopic tracking (U.S. Pat. No. 6,315,724), and accelerometers tracking (e.g. SENSR, Elkader, Iowa, USA, GPI 3 axis accelerometer and Gecko accelerometer).

In some embodiments, processor 487 receives location data (optionally comprising real time information about locations of scanning head 430 in real space and location of a body of subject in real space). Option processor 487 also receives the image data stream, optionally constituting actual image data from OCT engine 479 and/or other sources. In other words, processor 487 optionally receives information about what OCT engine 479 is imaging and where scanning head 430 was imaging it from. Combining information from these two sources (optionally in real time) produces information about the position of imaged objects (e.g. tissue features) with respect to a three-dimensional coordinate system. A collection of this data is referred to herein as 3D mapping. Processor 487 optionally combines of mapping data with other spatially distributed information, for example with historical information (for example from a previous scanning operation of a same tissue). The results are optionally displayable according to a variety of views for example over user interface 486.

Some embodiments comprise a probe positioning module. The positioning module optionally includes a servo mechanism optionally commendable by commands sent from processor 487 and/or serving to physically position scanning head 430 at a desired position.

User interface 486 optionally comprises tools for manipulating the display, behaviors of various parts of the system, operational parameters, and various other instructions to the system. Interface 486 also optionally provides probe placement instructions and/or feedback to a user using system-guided manual placement.

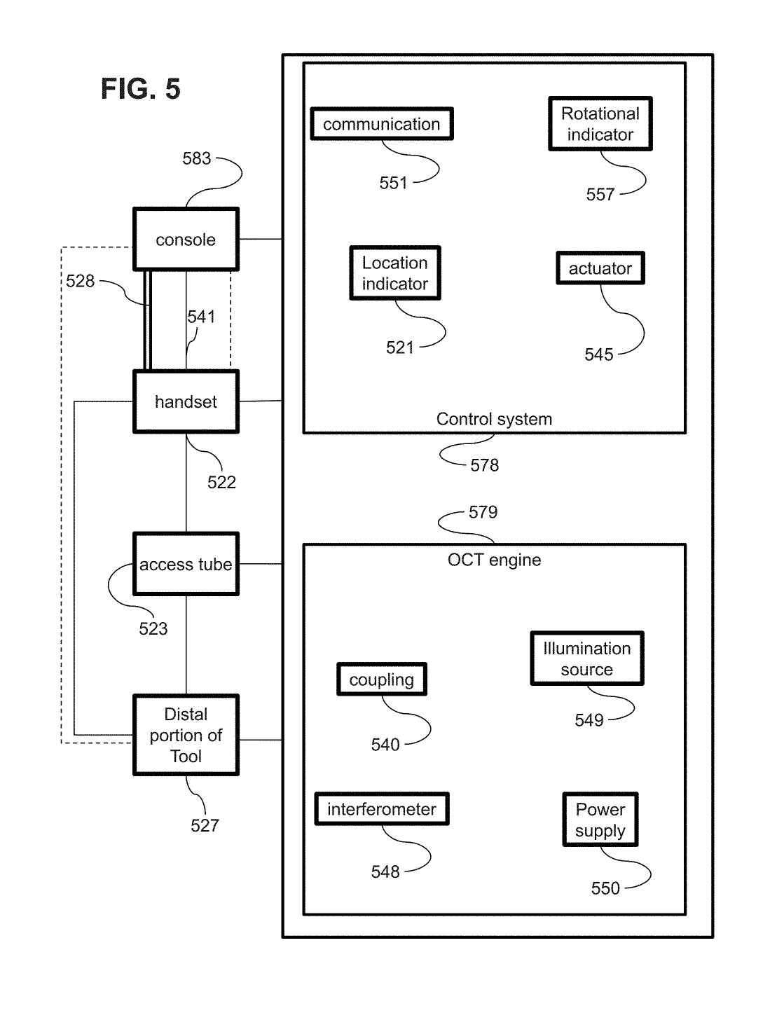

FIG. 5 is a high level block diagram illustrating optional geometries for placement of components of the OCT engine 579 and/or control system 578 in an OCT scanning system in accordance with some embodiments of the present invention. One some or all of the components of the OCT system may be housed for example in one or more of the following components: an external console 583 and/or a access tube 523 that is used as a port from outside a subject to a ROI and/or a handset 522 associated with a proximal end of a tool that remains outside the subject and/or a distal portion 527 of a tool that is sized and shaped to fit into access tube 523 and/or through access tube 523 to the ROI.

In some embodiments a tool may include handset 522 and distal portion 527. The handset may be connected to the distal portion by light guide including for example a shaft and/or an optical fiber OF. For example, the shaft and/or the OF may be part of a light guide leading from the handset to the distal portion of the invention. For example, the handset and/or light guide and/or distal portion may be permanently interconnected together. Optionally the interconnection may include a rotating joint (for example a rotating optic coupler). Optionally the tool including the OF, handset 522 and/or distal portion 527 may be fully retracted from access tube 523.

In some embodiments, an OCT engine 579 may include one some or all of an interferometer 548 and/or a power supply 550 and/or an illumination source 549 and/or a rotating optical coupler 540.

In some embodiments, control system 478 may include one, some or all of a communication means 551 (for example a wired connection and/or a wireless transceiver) and/or a rotational indicator 557 (indicating the rotation of the scanning head with respect to a reference for example access tube 523) and/or a localization indicator 550 (for example a 5 or 6 dimensional orientation sensor) and/or a actuator 545 for controlling the orientation and/or position of the scan head.

In some embodiments a tool may be connected to a consol via an optical fiber 528 and/or a wired connection 541 and/or a wireless connection (represented for example in FIG. 5 by dotted lines connecting handset 522 and/or distal portion 527 of the tool to consol 583.

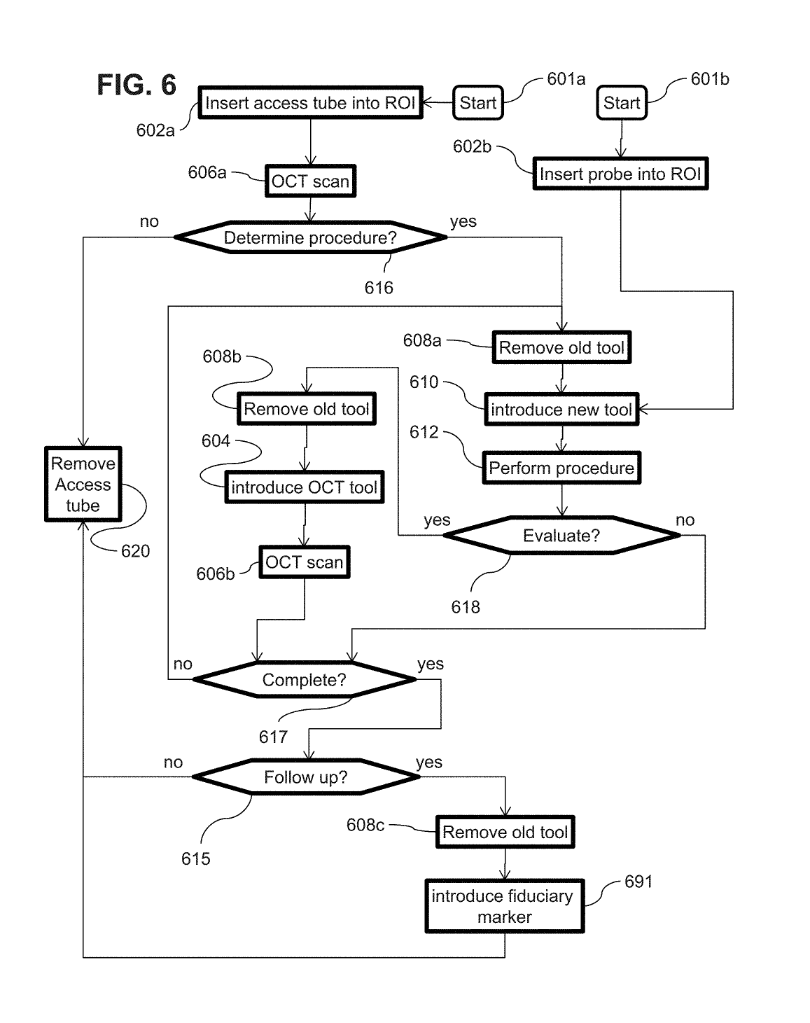

FIG. 6 is a flow chart illustration of various optional steps in a method of real time control and/or review of an internal procedure using an OCT tool in accordance with an embodiment of the present invention. For example, an access tube may be inserted 602 into a ROI and used for multiple procedures. For example procedures may optionally include OCT scanning. Optionally OCT scanning may be used to determine whether and/or what procedure is needed. Alternatively or addition OCT scanning may be used to determine whether a procedure was completed successfully.

In some embodiments an internal procedure may start 601a by inserting 602a a biopsy access tube to a ROI. Optionally the access tube may be used as a port for an OCT tool (for example using one or more of the methods or tools described herein above in the embodiments of FIGS. 1-4 and/or herein below). An OCT scan 606a may be performed. Scanning 606a may include 3D mapping of the scanned image. The results of the scan and or mapping may optionally be used to determine 616 whether to perform a further diagnostic procedure and/or an intervention in the ROI and/or where the intervention and/or diagnostic procedure should be focused. Once the necessary procedure has been determined 616 the OCT tool may be retracted 608a from the access tube and/or a new tool introduced 610.

In some embodiments, a ROI may have been identified that needs intervention. The procedure may alternatively start 601b by inserting 602b an access tube to a ROI and/or introducing 610 an intervention and/or diagnostic tool to the ROI.

In some embodiments a diagnostic procedure and/or a treatment may be performed 612. For example a diagnostic procedure may include measuring a temperature and/or taking an image (e.g. visible, infrared etc.). For example an intervention may include an ablation tool and/or an aspiration and/or ultrasonic treatment.

In some embodiments, OCT may be used to evaluate 618 the results of a procedure. For example an OCT scan may be used to evaluate 618 whether a lesion was completely ablated and/or removed. Alternatively or additionally an OCT scan may be used to evaluate 618 whether a sample was taken from the correct location. Use of OCT for evaluation may allow real time correction of procedures using a single access tube insertion. For example, evaluation 618 may include retracting 608b the intervention tool from the access tube and/or introducing 604 the OCT scanning tool. Then an OCT scan is optionally performed 606b (for example as described in any of the various embodiments herein above and/or herein below). Scanning 606b may include 3D mapping of the scanned image. Based on the OCT scan, the 3D mapping and/or based on other factors it may be decided 617 whether the necessary interventions and/or diagnostic procedures have been completed. Alternatively or additionally, the procedure may not use OCT to evaluate 618 the results of procedures performed.

In some embodiments it may be decided 615 that follow up procedures will be performed in the same ROI. For example, if follow-up is required, the OCT tool may be retracted 608c and/or the access tube may be used to introduce a 691 fiduciary marker into the ROI. Once all of the necessary procedures have been performed, the access tube is optionally removed 620 from the subject.

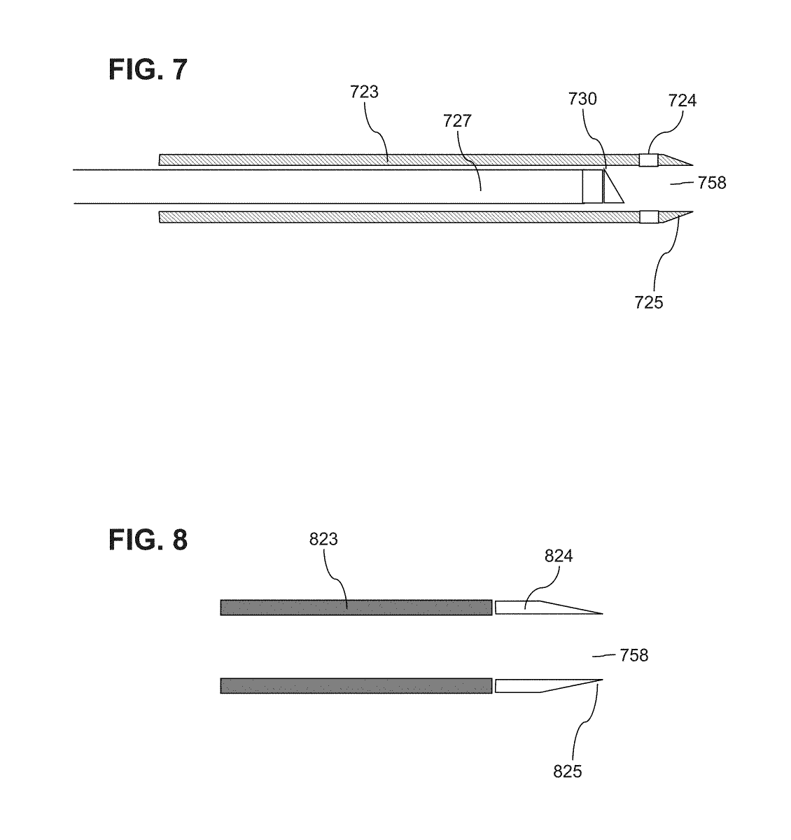

FIG. 7 is a cross sectional view of an OCT biopsy system including a multifunction single lumen access tube with a window near the distal end in accordance with an embodiment of the present invention. In some embodiments, multifunction access tube 723 includes a wrap around window 724 near the distal opening 758 of access tube 723. Optionally, window 724 is made of a material that conveys infrared light. Optionally window 724 is fused on its proximal end to a cannula leading for example to a proximal opening of access tube 723 and/or window 724 optionally fused on its distal side to a distal opening of the access tube and/or a sharp tip 725 of the cannula. Also illustrated in FIG. 7 is an optional OCT tool 727. For example, tool 727 includes a scanning head 730 for directing an OCT light beam through window 724 and/or directing reflected light coming back through window 724 up access tube 723 to a detector. Optionally head 730 may rotate inside of access tube 723, directing light along a azimuthal range.

In some embodiments access tube 723 may be inserted into a subject. Optionally sharp tip 725 may be used to puncture and/or penetrate the subject and/or tissue in a ROI. For example a obturator tip may be inserted into opening 758. For example, in some embodiments, the cannula and/or sharp tip 725 may be made of hard plastic and/or metal.

In some embodiments, the distance between the closest points of window 724 and opening 758 may range, for example, between 0.2 to 0.5 mm and/or between 0.5 to 2 mm and/or between 2-10 mm and/or between 10 to 40 mm and/or between 40 to 100 mm and/or between 100 to 200 mm. In some embodiments, the longitudinal distance (for example parallel to the axis of access tube 723) of window 724 (and/or of a set of windows) from its most distal point to its most proximal point may range for example between 0.5 to 1 mm and/or between 1 to 4 mm and/or between 4 to 15 mm and/or between 15 to 50 mm and/or between 50 mm to 200 mm.

FIG. 8 is a cross sectional view of an OCT biopsy system including a multifunction access tube with a window at the distal end in accordance with an embodiment of the present invention. In some embodiments, a window 824 may form all or part of the distal end of a access tube 823. For example, a distal end window 824 may have a sharpened tip 825 for puncturing and/or penetrating tissue. For example, distal tip window 824 may be made of a hard material such as sapphire and/or quartz. In some embodiments access tube 823 may be inserted into a subject. For example a obturator tip may be inserted into opening 758.

FIG. 9A is a cross sectional view of a multifunction access tube being used to place a fiducial marker in accordance with an embodiment of the present invention. Before or after use of access tube 723 for an OCT scan, access tube 723 may be used to place a fiducial marker 991. Optionally, marker 991 may be placed at a known spatial relation to an area scanned by the OCT system. For example marker 991 may be permanently and/or reversibly attached to an OCT scanning tool (for example tool 727). Optionally the access tube 723 may be used for introducing the OCT scan tool to the ROI and/or for placing fiducial marker 991. Optionally access tube 723 may be used for placement of marker 991 and/or OCT scanning with a single insertion of access tube 723 and/or while keeping access tube 723 in substantially the same position in the subject. For example, OCT scan tool 727 may scan through a distal window 724. Alternatively or additionally an OCT scan tool may be introduced into the ROI though a distal opening in the access tube. Optionally fiducial marker may be introduced into the ROI through distal opening 758 of access tube 723. For example fiducial marker 991 may include a passive marker and/or a gold marker and/or an active marker and/or a beacon marker.

FIG. 9B is a cross sectional view of a multifunction access tube with a distal location indicator in accordance with an embodiment of the present invention. In some embodiments, a location indicator may be positioned in the distal portion of a tool and/or an access tube. For example, a deploying marker 991 may be at a fixed relationship to OCT scanning head 730. While scanning head 730 is in use, marker 991 optionally indicates the location that is being scanned. Optionally, at some point in a procedure, marker 991 may be placed in a patient. For example, after scanning with OCT head 730, marker 991 may be placed in the subject. For example, marker 991 may be placed in a subject in a ROI where a structure of interest was identified by the OCT scan and/or marker 991 may be placed in a region where a follow up procedure may be necessary. Marker 991 optionally is connected to an applicator shaft 993 and/or cable. For example shaft 993 and/or a cable may be used to control the position of marker 991 and/or deploy marker 991. Alternatively or additionally shaft 993 and/or a cable may supply power and/or communication and/or command control to marker 991. In some embodiments, marker 991 and/or another tool may be used together with and/or separately and/or before and/or after an OCT tool and/or another tool.

FIG. 9C is a cross sectional view of a multifunction access tube being used to take a conventional biopsy in accordance with an embodiment of the present invention. Optionally, an OCT access tube and/or tool may be used in conjunction with other therapies and/or diagnostic procedures. For example, a tissue sampling tool 710 may be introduced through an access tube before and/or after and/or with an OCT scanning assembly.

FIG. 9D is a cross sectional view of a multifunction access tube with a core needle 997 in accordance with an embodiment of the present invention. For example, a tissue sampling tool 997 may be introduced through an access tube before and/or after and/or with an OCT scanning assembly.

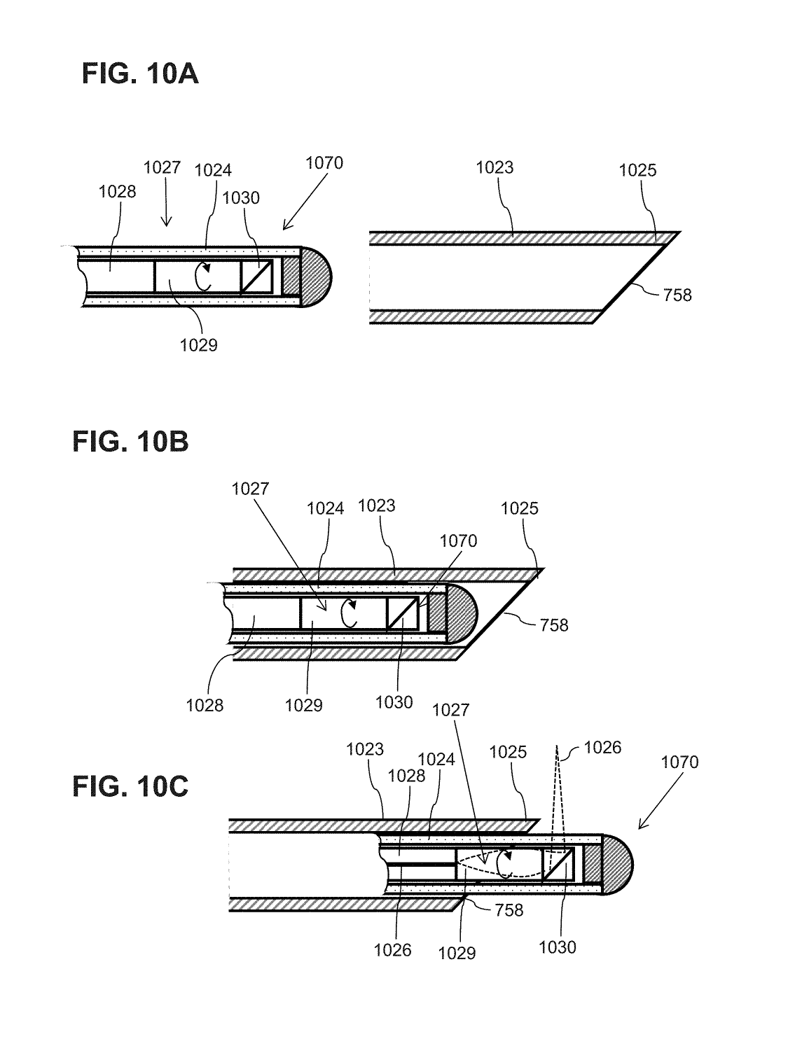

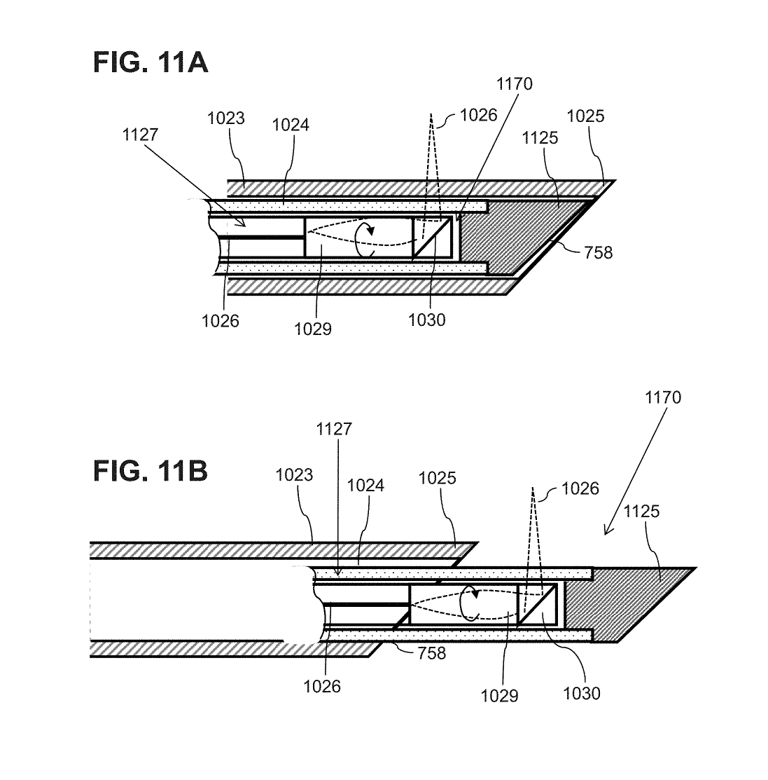

FIG. 10A-C are cross sectional views of a conventional single lumen access tube and an OCT tool head in accordance with an embodiment of the present invention. Optionally, access tube 1023 may be used with an internal core needle, and/or a harvesting tool. Optionally tool 1027 has a blunt tip. For example, the core needle may be introduced into access tube 1023 and the assembly (access tube with a sharp tip 1025 and core needle) inserted into the ROI. Subsequently, the core needle is optionally retracted and/or an OCT tool (for example tool 1027), is introduced into access tube 1023 (for example as illustrated in FIGS. 10A and 10B) and/or exposed to tissue in the ROI for example by inserting tool 1027 out distal opening 758 and/or retracting access tube 1023 such that tool 1027 remains in place and/or passes out opening 758 into the ROI (for example as illustrated in FIG. 10C).

In some embodiments an OCT tool 1027 may be introduced into and/or fully retracted out of access tube 1023. Optionally access tube may be used for other tools during a biopsy procedure according to the needs of a user, for example when biopsy tool 1027 has been retracted or before tool 1027 is introduced.

For example access tube 1023 may include an external cannula, having a 1.2 mm outer diameter (OD) and/or a 1.0 mm inner diameter (ID). Alternatively or additionally other size access tubes may be used. Optionally an OCT tool may have an OD of 0.9 mm to replace the core needle and/or be inserted into the lumen of access tube 1023. For access tubes of differing ID, the OD of an OCT may optionally be selected to fit.

In some embodiments an OCT tool 1027 includes a scan head 1030 (for example a prism which rotates inside a light conveying tube and/or window 1024) and/or an optical fiber OF 1028 and/or a GRIN 1029 (Gradient Index Lens). GRIN 1029 is optionally made of a suitable polymer, glass and/or other light conveying material at the desired wavelength (for example 0.8-1.7 Microns).

In some embodiments, scan head 1030 may redirect an axial beam of light out window 1024. Optionally scan head 1030 rotates causing beam 1026 to scan over an azimuthal angle. In FIG. 10C beam 1026 is shown redirected to substantially perpendicular to the axis of access tube 1023. Alternatively or additionally beam 1026 may be redirected at an angle between 80 to 90 degrees and/or between 60 to 80 degrees and/or between 30 to 60 degrees and/or between 5 to 30 degrees of the axis access tube 1023. Optionally beam 1026 is reflected back by tissue and/or the reflected beam is redirected by scan head 1030 back through a light guide to a detector. The light guide may include, for example by a GRIN 1029 and/or a shaft, for example OF 1028.

In some embodiments a distal section 1070 of tool 1027 may be extended out of the distal opening of the access tube (for example as illustrated in FIG. 10C). For example, distal section 1070 may include the scan head 1030 in a cavity (for example the lumen of a glass tube and/or for example in a lumen of plastic tube at a location of widow 1024). In the extended state the outer surface of distal section 1070 may be in contact with tissue of the ROI. Optionally the outer surface is stationary with respect to the tissue. In some embodiments, scan head 1030 may move (for example rotate with respect to the window and/or distal section 1070 and/or the tissue).

FIG. 11A is a cross sectional view of a conventional access tube and an embodiment of an OCT tool 1127 having a obturator tip 1125 in accordance with an embodiment of the present invention. Optionally access tube 1023 may be inserted into a tissue with tool 1127 inside the lumen (for example as illustrated in the configuration of FIG. 11A). Tool 1127 is optionally exposed to tissue in the ROI for example by inserting tool 1127 out distal opening 758 and/or retracting access tube 1023 such that tool 1027 remains in place and/or passes out opening 758 in the ROI (for example as illustrated in FIG. 11B).

In some embodiments a distal section 1170 of the tool 1127 may be extended out of the distal opening of the access tube (for example as illustrated in FIG. 11B). For example, distal section 1170 may include the scan head 1030 in a cavity (for example the lumen of a glass tube for example widow 1024). In the extended state the outer surface of distal section 1170 may be in contact with tissue of the ROI. Optionally the outer surface is stationary with respect to the tissue. In some embodiments, scan head 1030 may move (for example rotate with respect to the window and/or distal section 1170 and/or the tissue).

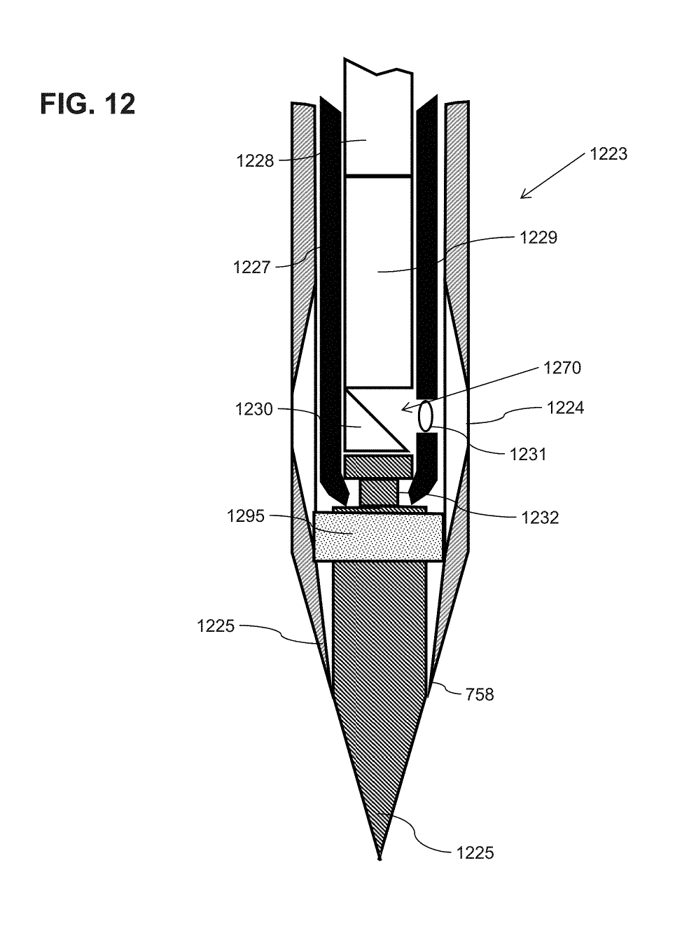

FIG. 12 is a cross sectional view of a rotating OCT tool with a non-rotating obturator tip in a multifunction OCT access tube in accordance with an embodiment of the present invention. A rotating inner tool 1227 optionally includes a FO 1228, GRIN 1229, scanning head 1270 and/or lens 1231. The distal end of tool 1227 is optionally attached to an obturator tip 1225. Optionally the connection between tool 1227 and tip 1225 includes a bearing 1232 which allows tool 1227 to rotate while tip 1225 remains stationary. For example, scan head 1270 may include a prism 1230 to redirect a beam through a window 1224 in access tube 1223. Optionally rotating scan head 1270 causes the beam to scan over an azimuthal angle.

In some embodiments, tip 1225 includes a stopper 1295 that presses against the stationary sharpened tip 1225 and/or walls of access tube 1223. For example stopper 1295 may be made of rubber and/or polymer. For example, stopper 1295 may seal distal opening 758 and/or protect tool 1227 from body liquids. Obturator tip 1225 optionally makes it possible to insert access tube 1223 and/or penetrate tissue with OCT tool 1227 in the lumen of the access tube. Optionally tool 1227 and/or stopper 1295 and/or obturator 1225 may be fully retracted from access tube 1223 while the access tube remains in place in the ROI. Optionally access tube 1223 may be used for alternative tools and/or procedures for example when tool 1227 is not in the lumen.

In some embodiments, a window may be fused to a sharp tip and/or a cannula of a access tube at a right angle (for example as illustrated in fusing of window 724 to access tube 723 and/or window 824 to access tube 823 in FIGS. 7 and 8 respectively). In some embodiments a portion of a window may be fused to a cannula and/or a sharp tip at an acute and/or obtuse (for example as illustrated in fusing of window 1224 to access tube 1223 in FIG. 12). For example the surface of fusing between the window and the access tube may be at an angle of between 0 to 10 degrees and/or 10 to 30 degrees and/or 30 to 60 degrees and/or 60 to 80 degrees and/or 80 to 85 degrees and/or 85 to 90 degrees to the axis of the access tube. Optionally the window may be connected with a form of mechanical interlocking, for example a screw thread and/or an interlocking tab and/or an o-ring and/or a press fit and/or an axial connector and/or a radial connector and/or a rotational connector. The window is optionally annular and/or forms a section of the cannula.

FIG. 13 is a cross sectional view of an OCT access tube with a distal scanning engine in accordance with an embodiment of the present invention. In some embodiments, an OCT tool includes a non-rotating FO 1328. Optionally, scanning may be performed by a rotating scanning head 1370. For example scanning head 1370 may include a reflecting element 1330 rotated by motor 1344 located in a distal portion of an access tube 1323. For example motor 1344 and/or element 1330 may be distal to FO 1328 and/or GRIN 1229. The access tube is optionally made of metal and/or other hard biocompatible plastics. Access tube 1323 may include a sharp tip 1325. For example sharp tip 1325 may not rotate with scanning head 1370. Optionally sharp tip 1325 is at the distal end of access tube 1323. Optionally, conducting wires may be included for the purpose of passing control signals and/or power to drive scan motor 1344. Alternative or additionally power and/or control signals may be delivered via conducting coating such as an ITO transparent conducting fine strip. Alternative or additionally motor 1344 may include a power supply (for example a battery) and/or control signals may be delivered via a wireless transmission. For example motor 1344 may include a micromotor available from Kinetron, The Netherlands and/or from Namiki of Japan. Optionally, micromotor 1344 includes speed control and/or stabilization.

Each of handsets illustrated in FIGS. 14-19 may optionally be used with any of the embodiments of OCT scanning tools described herein. Each of handsets illustrated in FIGS. 14-19 may optionally be used with any of the embodiments of access tubes described herein. Each of handsets illustrated in FIGS. 14-19 may optionally be permanently connected to an OCT scanning tool. Each of handsets illustrated in FIGS. 14-19 may optionally be fully retracted from an access tube while the tube remains in situ (for example with a distal end of the tube in a ROI). For example, fully retracting a handset and/or OCT scanning tool while the access tube remains in situ may facilitate use of the access tube for a different tool and/or procedures in the ROI without removing and/or reinserting the access tube. Alternatively or additionally each of handsets illustrated in FIGS. 14-19 may optionally reversibly attached to an OCT scanning tool. Alternatively or additionally each of handsets illustrated in FIGS. 14-19 may optionally permanently attached to an access tube.

FIG. 14 is a cross sectional view of an OCT system including an access tube, a tool and handset in accordance with an embodiment of the present invention. In some embodiments some and/or all parts of an OCT tool 1027 may be connected to a stationary consol, the access tube, the distal portion of the tool and/or the handset of the tool. For example, a consol may be connected to tool 1027 via a wire and/or an optical fiber and/or a wireless channel. For example, handset 1422 may include a localization sensor, an optical coupler (for example a rotating optical coupler), an actuator (for example a rotary actuator to rotate a scanning head and/or a linear actuator to position a sensor along a cannula), an illumination source, an interferometer, a radiation detector, a wired and/or wireless communication transceiver and/or a power source. For example an illumination source and/or detector may be mounted in a consol and/or connected to tool 1027 via an optical fiber for example reducing the number of components on tool 1027. Alternatively or additionally, the detector and/or illumination source may be located in the tool 1027 and/or handset 1422. Power may be supplied to tool 1027 via wires and/or communication between tool 1027 may be via a hardwired connection, for example increasing maneuverability of the tool. Alternatively or additionally, tool 1027 and/or handset 1422 may include a power source and/or a wireless transceiver, for example for un-tethered use.

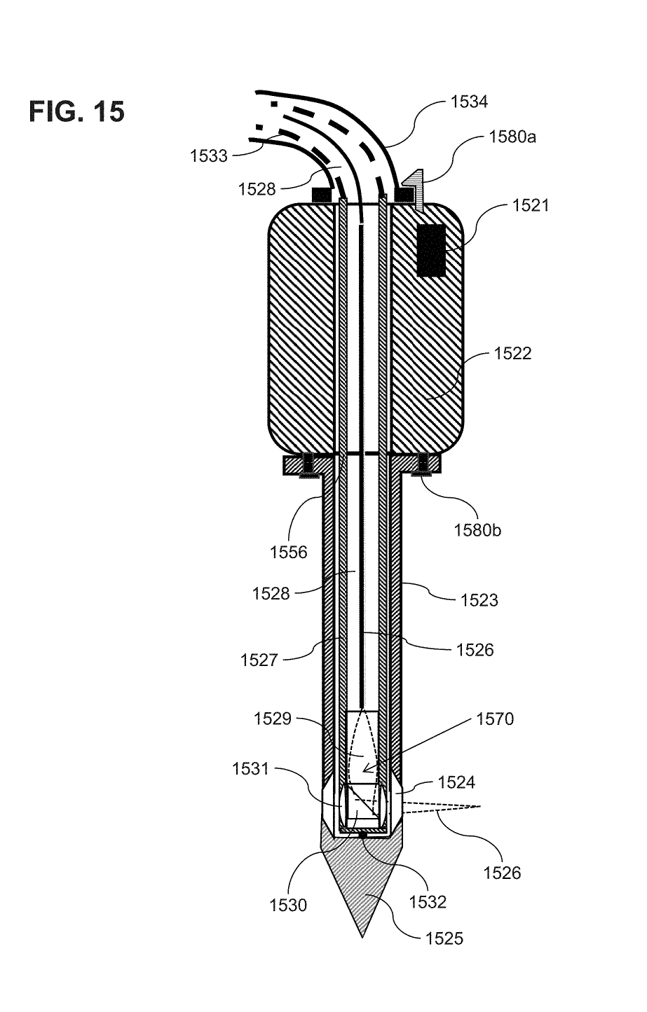

FIG. 15 is a cross sectional view of an OCT access tube and OCT tool handset including a localization sensor in accordance with an embodiment of the present invention. In some embodiments, an OCT tool handset 1522 includes a localization indicator 1521. For example, indicator 1521 may include a sensor that outputs to a consol information on location and/or orientation in 5 or 6 degrees of freedom. Optionally, the output of indicator 1521, is sent to a consol, for example over a wired connection and/or a wireless connection. A processor in the consol optionally tracks the position and/or orientation of a scanning head 1570 (for example including a beam directing assembly) at the distal end of the tool.

In some embodiments non-rotating handset 1522 supports a rotating element 1527. Element 1527 supports for example a two way fiber optic cable, FO 1528. In some embodiments, element 1527 is connected on its proximal end to a flexible cable 1533 which is optionally connected to and/or rotated by a consol (not shown). The distal end of element 1527 optionally includes scanning head 1570. For example scanning head 1570 may include a gradient index lens, GRIN 1529, a prism 1530, and/or one or more lenslets 1531. Optionally, in the exemplary embodiment of FIG. 15, one lenslet 1531 is used for focusing beam 1526 while the other lenslet is for balancing purposes. An outer non rotating sheath 1534 optionally includes electrical conductors.

In some embodiments, handset 1522 is rotationally coupled to a rotating element 1527. The distal end of rotating element 1527 optionally includes beam directing scanning head 1570 and/or fits rotatingly into a cannula of access tube 1523. An optional bearing 1532 (for example a trust bearing and/or ball) supports the distal end of rotating element 1527. For example bearing 1532 may reduce vibrations when element 1527 is rotated. In some embodiments element 1527 is reversibly secured to the proximal opening 1556 of handset 1522 by a latch 1580a.

In some embodiments rotating assembly 1527 may rotate independent of access tube 1523. Optionally access tube 1523 may be disposable and/or single use. Alternatively or additionally, access tube 1523 may be reusable and/or sterilizable. The proximal end of access tube 1523 includes an optional by fast lock mechanism including for example bayonet pins 1580b. For example the fast lock mechanism may reversibly connect access tube 1523 to handset 1522.

In some embodiments, the distal end of access tube 1523 includes sharp tip, for example a obturator 1525. Obturator 1525 is optionally used to insert the distal end of access tube 1523 into soft tissue of a subject. The distal end of access tube 1523 optionally includes a window 1524 which may for example consist of a light conveying ring. Optionally window 1524 is bonded to obturator 1525.

In some embodiments, the rotational orientation of rotational assembly 1527 is measured by a location sensor on the rotational assembly. Alternatively or additionally, a sensor may measure the relative orientation of rotational assembly with respect to handset 1522. For example, there may be optical and/or physical and/or electronic markers on rotational assembly 1527 that are detected by a sensor on handset 1522 and/or there may be optical and/or physical and/or electronic markers on handset 1522 that are detected by a sensor on rotational assembly 1527. Alternatively or additionally, a sensor may measure the relative orientation of rotational assembly with respect to access tube 1523. For example, there may markers on widow 1524 that are detected by their effect on beam 1526.

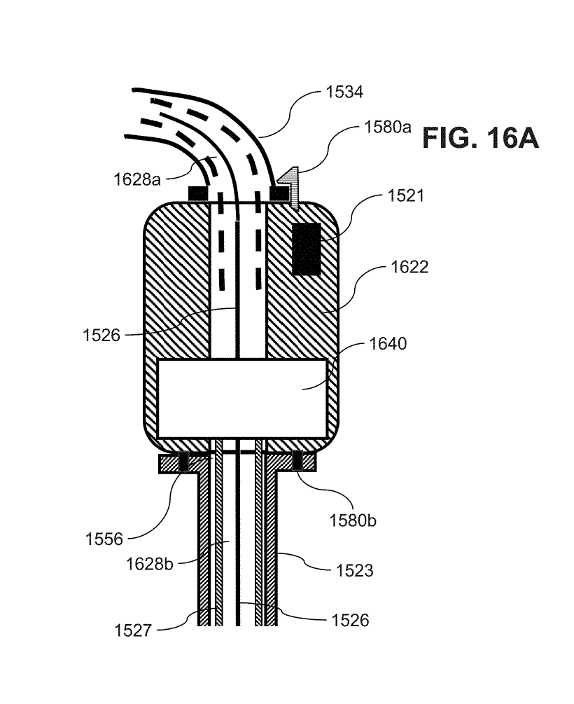

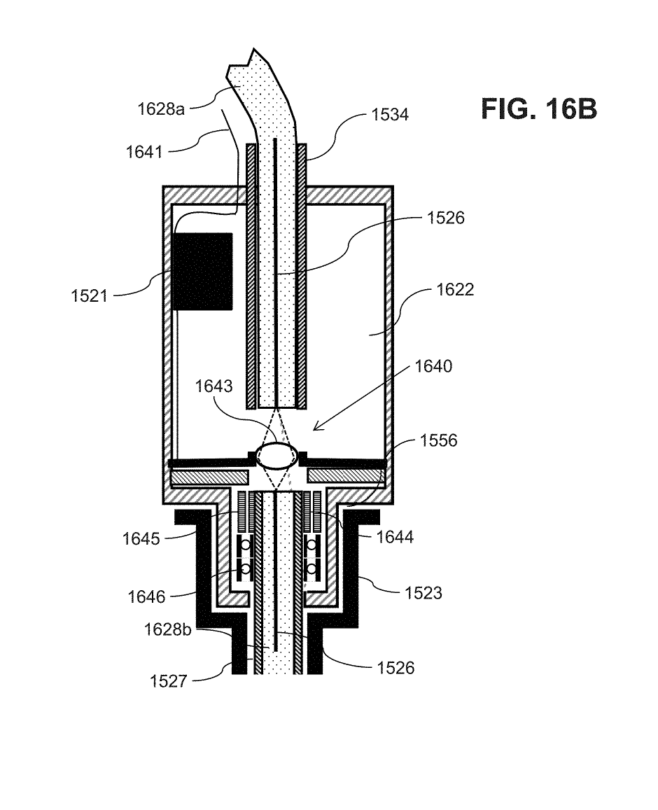

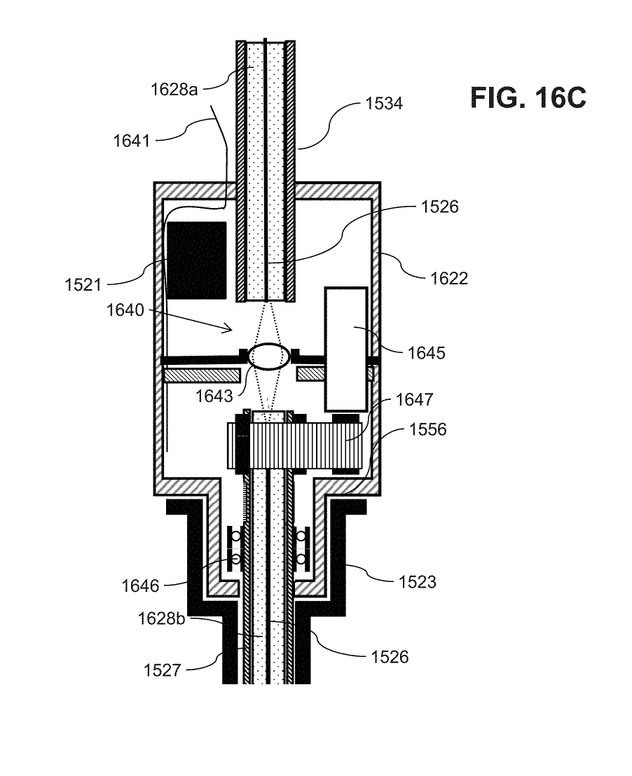

FIG. 16A-C is a cross sectional view of an OCT tool handset including a localization sensor and a rotational coupler in accordance with an embodiment of the present invention. In some embodiments a handset 1622 may include an optical rotary joint that makes it possible to rotate rotation assembly 1527 independently of any connection to the consol, for example cable 1534 and/or optical fiber 1628a. Optionally, an optical fiber 1628b is permanently connected to handset 1622 via a rotating optical couple 1640. For example, decoupling rotation of scanning head 1570 from the consol may make it easier for an operator of the OCT device to control the position scanning head 1570 and/or the focus of the OCT apparatus. In some embodiments, using an external interferometer may allow use of a more precise instrument that is not exposed to movement and/or changing environmental conditions of the handset and/or may reduce the weight of the handset in comparison to the including an interferometer in the handset.

In some embodiments, optical two way FO cable 1628a is non rotating. For example cable 1628a may carry a light from an OCT engine (for example including an interferometer and/or an illumination source) to the OCT tool and/or cable 1628a may bring a reflected light signal from the OCT tool to a sensor. The sensor and/or the OCT engine may be located in a console (not shown). The optical signal is optionally transferred from the stationary FO 1628a to the rotating FO 1628b and back via a coupler 1640. For example coupler 1640 may include coupling lenses. For example an optical coupler may include a ZJ2-155-28 coupler available from Princetel Inc., 1595 Reed Rd. Pennington, N.J. 08619(609) 895-9890. Optionally, the distal portion of the OCT apparatus may be the same as that illustrated in FIG. 15. For example, rotating assembly 1527 may supported by a bearing 1532 as illustrated in FIG. 15. Alternatively or additionally, handset 1622 may be used with another OCT tool, for example a retractable OCT tool. Examples of retractable OCT tools are illustrated above for example in FIGS. 10A-10B, 11A-11B, 12 and 14.

In some embodiments, rotating assembly 1527 can be rotated with respect to handset 1622 by an actuator, for example a DC motor and/or a stepper motor (for example as illustrated in FIGS. 16B and 16C) and/or a manual device. Optionally the actuator may be housed inside handset 1622.

FIG. 16B illustrates schematically details of handset 1622 with direct drive hollow shaft brush less motor for controlling rotation of rotational assembly 1527 in accordance with an embodiment of the present invention. For example, handset 1622 may include coupling optics 1643 (for example a set of lenses). The direct drive stepper motor optionally includes a rotor 1644 and/or a stator 1645 and/or bearing 1646 and/or a corresponding control circuit as is known in the art. In some embodiments a hard wired connection 1641 may be used for electrical communication with a consol. For example wired connection 1641 may carry control signals from a portable user interface to the consol and/or connection 1641 may carry sensor output from location indicator 1521 to the consol and/or connection 1641 may carry power maybe electrical power and/or control signals from consol to the OCT tool (for example signals and/or power may control the stepper motor). Alternatively or additionally, handset 1622 may include a wireless connection to a consol and/or an internal power source (for example a battery).

FIG. 16C illustrates schematically details of handset 1622 with belt drive brush less motor for controlling rotation of rotational assembly 1527 in accordance with an embodiment of the present invention. For example, a brushless motor 1645 may rotate assembly 1527 via a transmission 1647.

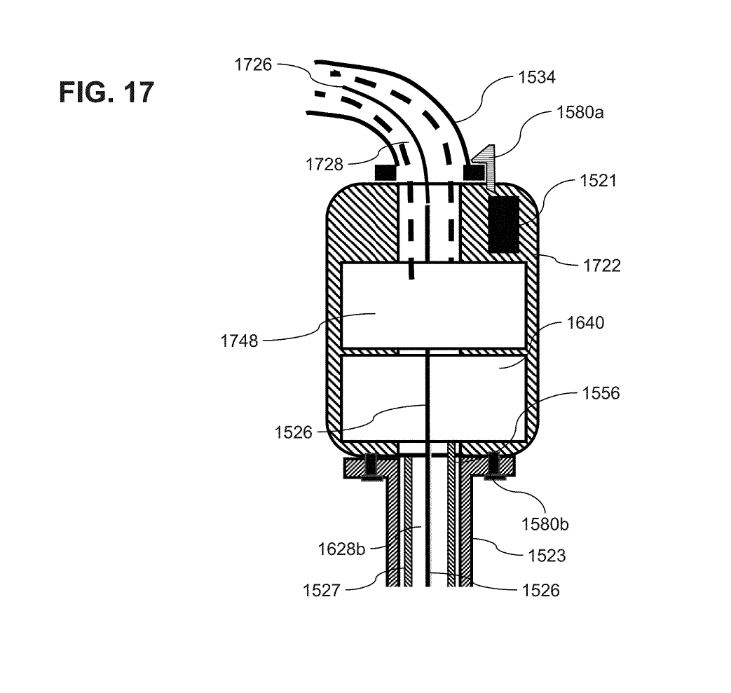

FIG. 17 is a cross sectional view of an OCT tool handset including a localization sensor, a rotational coupler and an interferometer in accordance with an embodiment of the present invention. Optionally illumination 1726 is provided from an external source (for example a consol) via stationary one way FO 1728.

In some embodiments, an interferometer 1748 may be fiber or prism based. Interferometer 1748 may include for example an interference signal generator and/or a detector. Providing the interferometer in the handset may in some embodiments reduce detection noise with respect to embodiments with an external interferometer, for example because the signal does not pass through a long FO before reaching the detector. Use of an external illumination source may in some embodiments have an advantage that a larger powerful and/or precisely controlled illumination source may be used without adding weight to the handset.

In some embodiments a hard wired connection may be used for electrical communication with a consol. For example a wired connection 1641 may carry control signals from a portable user interface to the consol and/or connection 1641 may carry sensor output from location indicator 1521 and/or interferometer 1748 to the consol and/or the wired connection may carry power, for example electrical power and/or control signals from consol to the OCT tool. Alternatively or additionally, handset 1722 may include a wireless connection to a consol and/or an internal power source (for example a battery).

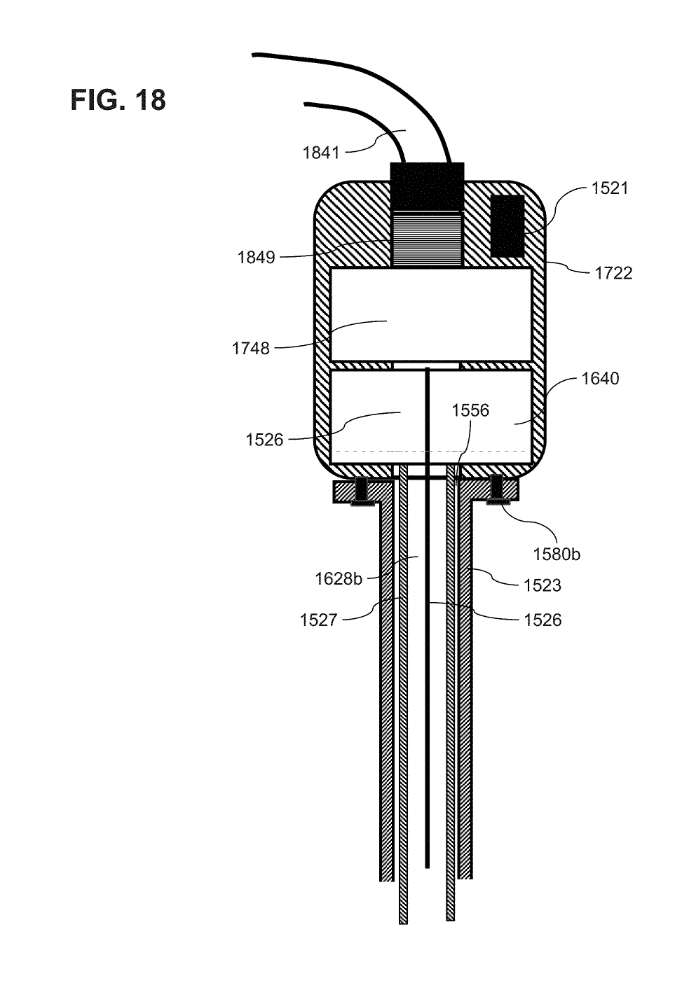

FIG. 18 is a cross sectional view of an OCT tool handset including a localization sensor and all of the optical components, for example a rotational coupler 1640, an interferometer 1748 and/or a light source 1849 in accordance with an embodiment of the present invention. Providing all of the optical components in the handset allows the OCT to be free of external FO connections. This may make it easier to maneuver the tool. Optionally a wired connection 1841 may supply power and/or communication links to external sources. Alternatively or additionally, wired connection 1841 may supply power only and/or a wireless communication system may be supplied for control messages and/or data transfer. In some embodiments, using wireless communication would allow the tool to be used without a hard wired connection to a consol further increasing maneuverability.

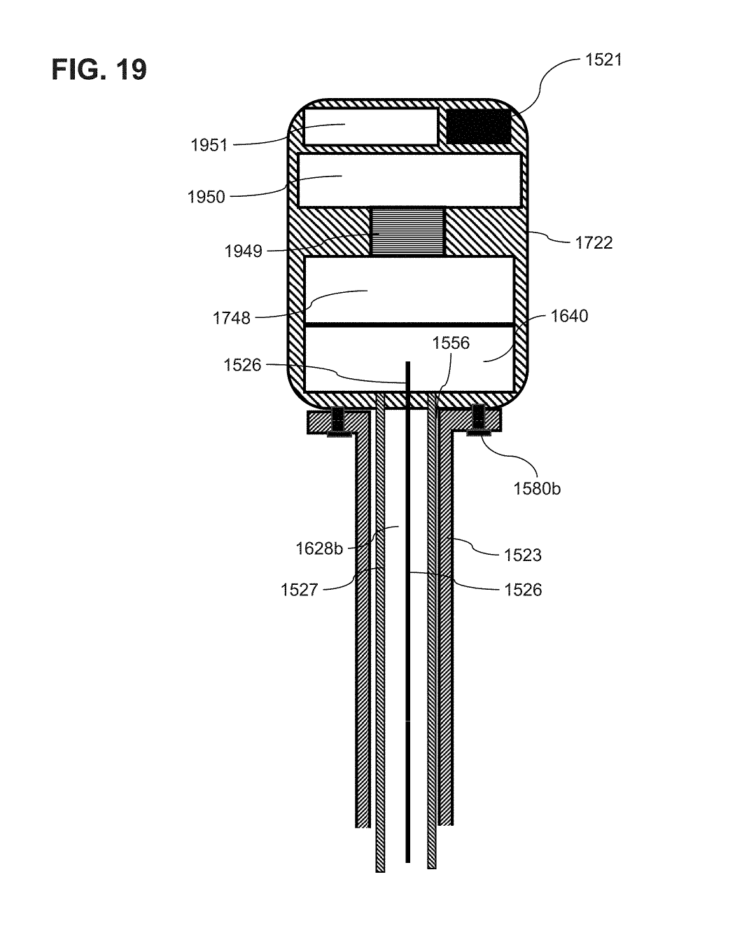

FIG. 19 is a cross sectional view of a wireless OCT tool handset including for example a localization sensor 1521, a rotational coupler 1640, an interferometer 1748 and a light source 1949, a power source 1950 and a wireless transmitter 1951 in accordance with an embodiment of the present invention. Including power source 1950 and light source 1949 in the handset optionally makes the handset untethered allowing the operator additional maneuverability when working.

It is expected that during the life of a patent maturing from this application many relevant technologies (for example diagnostic techniques and/or imaging techniques and/or optical scanning techniques) will be developed and the scope of the terms is intended to include all such new technologies a priori.

As used herein the term “about” refers to ±5%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-30. (canceled)

31. A scanning OCT system comprising:
a biopsy access tube including rigid cannula of between 7G to 25G including a lumen with a proximal opening;
a tool including:
a light guide sized to fit into the lumen of said biopsy access tube,
a scanning head sized to fit within said lumen; said scanning head light attached to a distal end of said light guide, said scanning head directing a light beam from outside said access tube to said light path, said scanning head being adjustable to redirect light from different azimuthal angles with respect to said access tube, and
a location indicator having a fixed spatial orientation with respect to said scanning head and retractable from said access tube with said scanning head.

32. The system of claim 31, wherein said tool is fully retractable from said access tube.

33. The system of claim 31, further comprising:
a GRIN lens between said light scanning head and said light guide.

34. The system of claim 31, further comprising:
an interferometer permanently fixed in orientation with respect to a proximal end of said light guide.

35. The system of claim 31, further comprising:
an OCT engine receiving said light from a proximal end of said light guide;
a processor for computing a 3D image map from said OCT engine and said location indicator.

36. The system of claim 35, further including:
a wireless transceiver for conveying data from said OCT to said processor.

37. The system of claim 36, further comprising:
a rotational optical coupler conveying said light from said light guide to said OCT engine.

38. The system of claim 31, wherein said location indicator retractable from said access tube with said light directing element.

39. The system of claim 31, wherein said location indicator includes a position sensor with at least 5 degrees of freedom.

40. The system of claim 31, wherein said location indicator includes fiduciary marker.

41. The system of claim 31, further including a processor to compute a location of a source of said light based on a location of said location indicator.

42-50. (canceled)

51. The system of claim 31, wherein said biopsy access tube further comprises:
a distal opening;
a window in said cannula between said proximal opening and said distal opening, said window including a transparent pane preventing contact between said light directing element and said tissue.

52. The system of claim 51, wherein said biopsy access tube further comprises:
a light directing element slidably fitting into said lumen from said proximal opening to said window, said light directing element redirecting a beam to a light guide leading to said proximal opening;
a plug mounted distally to said light directing element, said plug sliding axially with said cannula, said plug blocking fluid communication between said distal opening and said light directing element.

53. The system of claim 52, wherein said pane, said cannula and said plug prevent fluid communication between said light directing element and an exterior of said cannula.

54. The system of claim 52, further comprising:
a rotational connector between said light directing element and said plug allowing said plug to rotate around an axis of the cannula independently of said plug.

54. The system of claim 31, wherein said light guide includes an optical fiber.

55. The system of claim 31, wherein said location indicator includes a position sensor with at least 5 degrees of freedom.
